(19) United States
(12) Patent Application Publication
(10) Pub. No.: US 2025/0034609 A1
(43) Pub. Date: Jan. 30, 2025

(54) HIGH PERFORMANCE ALPHA-AMYLASES FOR STARCH LIQUEFACTION

(71) Applicant: DANISCO US INC., Palo Alto, CA (US)

(72) Inventors: Veli ALKAN, Oegstgeest (NL); Marc KOLKMAN, Oegstgeest (NL); Frank KOOPMAN, Oegstgeest (NL); Sang-kyu LEE, Palo Alto, CA (US); Chris LEEFLANG, Oegstgeest (NL); Kefeng NI, Shanghai (CN); Sina PRICELIUS, Oegstgeest (NL); Zhen QIAN, Palo Alto, CA (US); Zhongmei TANG, Shanghai (CN); Marco VAN BRUSSEL-ZWIJNEN, Oegstgeest (NL)

(21) Appl. No.: 18/710,653

(22) PCT Filed: Nov. 18, 2022
(86) PCT No.: PCT/US2022/050353

§ 371 Date: May 16, 2024

Related U.S. Application Data
(60) Provisional application No. 63/280,891, filed on Nov. 18, 2021.

(51) Int. Cl. C12P 19/02; C12N 9/26

(52) U.S. Cl. CPC C12P 19/02; C12N 9/2414; C12Y 302/01001

(57) ABSTRACT

Disclosed are compositions and methods relating to engineered α-amylases. The engineered α-amy lases outperform commercial combinatoral variant α-amylases, which are currently the industry standard. The engineered α-amylases are useful for starch liquefaction and saccharification, and may also be useful for cleaning starchy stains, textile desizing, baking, and brewing.

### CROSS REFERENCE TO RELATED APPLICATIONS

This application claims the benefit of U.S. Provisional Application No. 63/280,891, filed Nov. 18, 2021, which is hereby incorporated by reference in its entirety,

### FIELD OF THE INVENTION

Disclosed are compositions and methods relating to engineered α-amylases designed for efficient starch liquefaction. The engineered α-amylases outperform commercial combinatoral variant α-amylases, which are currently the industry standard. The engineered α-amylases are useful for starch liquefaction and saccharification, and may also be useful for cleaning starchy stains, textile desizing, baking, and brewing.

### BACKGROUND

Starch consists of a mixture of amylose (15-30% w/w) and amylopectin (70-85% w/w). Amylose consists of linear chains of α-1,4-linked glucose units having a molecular weight (MW) from about 60,000 to about 800,000. Amylopectin is a branched polymer containing α-1,6 branch points every 24-30 glucose units; its MW may be as high as 100 million.

Sugars from starch, in the form of concentrated dextrose syrups, are currently produced by an enzyme catalyzed process involving: (i) gelatinization and liquefaction (or viscosity reduction) of solid starch with an α-amylase into dextrins having an average degree of polymerization of about 7-10 and (ii) saccharification of the resulting liquefied starch (i.e. starch hydrolysate) with glucoamylase. The resulting syrup has a high glucose content. Much of the glucose syrup that is commercially produced is subsequently enzymatically isomerized to a dextrose/fructose mixture known as isosyrup. The resulting syrup also may be fermented with microorganisms, such as yeast, to produce commercial products including ethanol, citric acid, lactic acid, succinic acid, itaconic acid, monosodium glutamate, gluconates, lysine, other organic acids, other amino acids, and other biochemicals, for example. Fermentation and saccharification can be conducted simultaneously (i.e., via a simultaneous saccharification and fermentation (SSF) process) to achieve greater economy and efficiency.

α-amylases hydrolyze starch, glycogen, and related polysaccharides by cleaving internal α-1,4-glucosidic bonds at random. α-amylases, particularly from Bacilli, have been used for a variety of different purposes, including starch liquefaction and saccharification, textile desizing, starch modification in the paper and pulp industry, brewing, baking, production of syrups for the food industry, production of feedstocks for fermentation processes, and in animal feed to increase digestability. These enzymes can also be used to remove starchy soils and stains during dishwashing and laundry washing.

Numerous publications have described single mutations and multiple (i.e., combinatorial) mutations in α-amylases. However, the need exists for ever-more-robust engineered α-amylases molecules that out perform thos made using conventional strategies.

### SUMMARY

The present compositions and methods relate to engineered α-amylase polypeptides, and methods of use, thereof. Aspects and embodiments of the present compositions and methods are summarized in the following separately-numbered paragraphs:
1. In a first aspect, a non-naturally-occuring engineered α-amylase is provided, having at least 85% amino acid sequence identity relative to SEQ ID NO: 1, SEQ ID NO: 2, SEQ ID NO: 3 and/or SEQ ID NO: 4, and having α-amylase activity.
2. In some embodiments, a nucleic acid encoding the non-naturally-occuring engineered α-amylase of paragraph 1 is provided.
3. In some embodiments, an expression vector comprising the nucleic acid of paragraph 2 is provided.
4. In some embodiments, a cell comprising the expression vector of paragraph 3 is provided.
5. In some embodiments, a cell expressing the non-naturally-occuring engineered α-amylase of paragraph 1 is provided.
6. In some embodiments, a formulated composition comprising the non-naturally-occuring engineered α-amylase of paragraph 1 is provided.
7. In some embodiments, a method for saccharifying a composition comprising starch to produce a composition comprising glucose is provided, wherein the method comprises: (i) contacting the solution comprising starch with effective amount of the variant amylase of any of the paragraphs 1; and (ii) saccharifying the solution comprising starch to produce the composition comprising glucose; wherein the variant amylase catalyzes the saccharification of the starch solution to glucose.

These and other aspects and embodiments of the compositions and methods will be apparent from the present description and drawings.

### BRIEF DESCRIPTION OF THE DRAWINGS

FIG. 1 is a Clustal W amino acid sequence alignment of engineered α-amylases 1˜4 and the naturally-occuring α-amylases from Bacillus lichenifomis and Bacillus stearothermophilus.

### DETAILED DESCRIPTION

### 1. Introduction

Described are compositions and methods relating to engineered α-amylase enzymes that exhibit increased high temperature liquefaction performance at low pH in the absence of additional stabilizing agents such as calcium and sodium ions. The engineered α-amylases demonstrated 50-90% residual activity at pH 5, 30-70% activity at pH 4.8, and 10-35% activity at pH 4.5, after a short incubation at 110° C. for 7-9 minutes, followed by a 2 hr incubation at 95° C.

The engineered α-amylases are demonstrably useful for starch liquefaction and saccharification, but are likely also useful for cleaning starchy stains in laundry, dishwashing, and other applications, for textile processing (e.g., desizing), in animal feed for improving digestibility, and and for baking and brewing. These and other aspects of the compositions and methods are described in detail, below.

### 2. Definitions and Abbreviations

Prior to describing the various aspects and embodiments of the present engineered α-amylases and methods of use, thereof, the following definitions and abbreviations are described.

Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are provided below.

### 2.1. Abbreviations and Acronyms

The following abbreviations/acronyms have the following meanings unless otherwise specified:
DE dextrose equivalent
DNA deoxyribonucleic acid
ds or DS dry solids
EC Enzyme Commission
EOF end of fermentation
GA glucoamylase
GAU/g ds glucoamylase activity unit/gram dry solids
HFCS high fructose corn syrup
MW molecular weight
ppm parts per million, e.g., μg protein per gram dry solid
SSF simultaneous saccharification and fermentation
SSU/g solid soluble starch unit/gram dry solids
sp. species
Tm melting temperature
w/v weight/volume
w/w weight/weight
v/v volume/volume
wt % weight percent
° C. degrees Centigrade
H₂O water
DI deionized water
g or gm grams
mg milligrams
kg kilograms
mL and ml milliliters
mm millimeters
mM millimolar
M molar
U units
sec seconds
min(s) minute/minutes
hr(s) hour/hours
ETOH ethanol
eq. equivalents
Tris-HCl tris(hydroxymethyl)aminomethane hydrochloride
μg/gds μg enzyme protein per gram corn starch dry solid
nd not detectable

### 2.2. Definitions

The terms “amylase” or “amylolytic enzyme” refer to an enzyme that is, among other things, capable of catalyzing the degradation of starch. α-amylases are hydrolases that cleave the α-D-(1-+4) O-glycosidic linkages in starch. Generally, α-amylases (EC 3.2.1.1; α-D-(1-+4)-glucan glucanohydrolase) are defined as endo-acting enzymes cleaving α-D-(1-+4) O-glycosidic linkages within the starch molecule in a random fashion yielding polysaccharides containing three or more (1-4)-α-linked D-glucose units. In contrast, the exo-acting amylolytic enzymes, such as β-amylases (EC 3.2.1.2; α-D-(1-+4)-glucan maltohydrolase) and some product-specific amylases like maltogenic α-amylase (EC 3.2.1.133) cleave the polysaccharide molecule from the non-reducing end of the substrate. β-amylases, α-glucosidases (EC 3.2.1.20; α-D-glucoside glucohydrolase), glucoamylase (EC 3.2.1.3; α-D-(1->4)-glucan glucohydrolase), and product-specific amylases like the maltotetraosidases (EC 3.2.1.60) and the maltohexaosidases (EC 3.2.1.98) can produce malto-oligosaccharides of a specific length or enriched syrups of specific maltooligosaccharides.

The term “starch” refers to any material comprised of the complex polysaccharide carbohydrates of plants, comprised of amylose and amylopectin with the formula (C6H10O5)x, wherein X can be any number. The term includes plant-based materials such as grains, cereal, grasses, tubers and roots, and more specifically materials obtained from wheat, barley, corn, rye, rice, sorghum, brans, cassava, millet, milo, potato, sweet potato, and tapioca. The term “starch” includes granular starch. The term “granular starch” refers to raw, i.e., uncooked starch, e.g., starch that has not been subject to gelatinization.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “engineered” refers to a molecule that has been modified using any number of different methods that, in combination, involve a holistic approach to protein modification that is more complex than making single or combinatorial mutations. Engineered proteins may be essentially unrecognizable from any particular “parent” molecule and are therefore difficult to characterize as “variants.”

In the case of the present α-amylases, “activity” refers to α-amylase activity, which can be measured as described, herein.

The term “performance benefit” refers to an improvement in a desirable property of a molecule. Exemplary performance benefits include, but are not limited to, increased hydrolysis of a starch substrate, increased grain, cereal or other starch substrate liquifaction performance, increased cleaning performance, increased thermal stability, increased detergent stability, increased storage stability, increased solubility, an altered pH profile, decreased calcium dependence, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

The terms “protease” and “proteinase” refer to an enzyme protein that has the ability to perform “proteolysis” or “proteolytic cleavage” which refers to hydrolysis of peptide bonds that link amino acids together in a peptide or polypeptide chain forming the protein. This activity of a protease as a protein-digesting enzyme is referred to as “proteolytic activity.” Many well-known procedures exist for measuring proteolytic activity (See e.g., Kalisz, “Microbial Proteinases,” In: Fiechter (ed.), Advances in Biochemical Engineering/Biotechnology, (1988)).

“Combinatorial variants” are variants comprising two or more mutations, e.g., 2, 3, 4, 5, 6, 7, 8, 9, 10, or more, substitutions, deletions, and/or insertions.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an amylase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t½) given in minutes, hours, or days, during which half the enzyme activity is lost under defined conditions. The half-life may be calculated by measuring residual α-amylase activity following exposure to (i.e., challenge by) an elevated temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may contain chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tm), where one half of the hybridized nucleic acids are unpaired with the complementary strand. Mismatched nucleotides within the duplex lower the Tm. A nucleic acid encoding an α-amylase may have a Tm reduced by 1° C.-3° C. or more compared to a duplex formed between the nucleotide of SEQ ID NO: 2 and its identical complement.

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “water hardness” is a measure of the minerals (e.g., calcium and magnesium) present in water.

“A cultured cell material comprising an amylase” or similar language, refers to a cell lysate or supernatant (including media) that includes an amylase as a component. The cell material may be from a heterologous host that is grown in culture for the purpose of producing the amylase.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either termini are included. For example, a protein with five amino acid deletions of the C-terminus of the mature engineered α-amylases of SEQ ID NOs: 1-4 would have a percent sequence identity of about 99% (612/617 identical residues×100, rounded to the nearest whole number) relative to the original polypeptidse. Such truncated polypeptides would be encompassed by the language “at least 99% amino acid sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particulary Pezizomycotina species.

The term “degree of polymerization” (DP) refers to the number (n) of anhydro-glucopyranose units in a given saccharide. Examples of DP1 are the monosaccharides glucose and fructose. Examples of DP2 are the disaccharides maltose and sucrose. The term “DE,” or “dextrose equivalent,” is defined as the percentage of reducing sugar, i.e., D-glucose, as a fraction of total carbohydrate in a syrup.

The term “dry solids content” (ds) refers to the total solids of a slurry in a dry weight percent basis. The term “slurry” refers to an aqueous mixture containing insoluble solids.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

An “ethanologenic microorganism” refers to a microorganism with the ability to convert a sugar or oligosaccharide to ethanol.

The term “fermented beverage” refers to any beverage produced by a method comprising a fermentation process, such as a microbial fermentation, e.g., a bacterial and/or fungal fermentation. “Beer” is an example of such a fermented beverage, and the term “beer” is meant to comprise any fermented wort produced by fermentation/brewing of a starch-containing plant material.

The term “malt” refers to any malted cereal grain, such as malted barley or wheat.

The term “adjunct” refers to any starch and/or sugar containing plant material that is not malt, such as barley or wheat malt. Examples are well known in the art and widely used in specialty fermented products and in cheeper beers.

The term “mash” refers to an aqueous slurry of any starch and/or sugar containing plant material, such as grist, e.g., comprising crushed barley malt, crushed barley, and/or other adjunct or a combination thereof, mixed with water later to be separated into wort and spent grains.

The term “wort” refers to the unfermented liquor run-off following extracting the grist during mashing.

The term “about” refers to ±15% to the referenced value.

### 3. Engineered α-Amylases

The present compositions and methods are based on engineered α-amylase enzymes that out-perforn in industrial applications conventional α-amylase variants that include either single mutations or combinations of mutations. The engineered molecules were created from the combined knowledge and expereince of protein scientists and the use of advanced computer analysis. As such, the engineered α-amylases are difficult to characterize as variants of a parent molecule, and better characterized as new, non-naturally-occuring molecules.

Four engineered α-amylases are described and tested, herein, that have in common greater than 85% amino acid sequence identity. The molecules represent an optimization of the Carbohydrate-Active Enzymes database (CAZy) Family 13 amylases, and similarly, any amylase that has heretofore been referred to by the descriptive term, “Termamyl-like.” Examples of such α-amylases are those from Bacillus spp. Such as B. lichenifomis (i.e., BLA and LAT), B. stearothermophilus (i.e., BSG), and B. amyloliquifaciens (i.e., P00692, BACAM, and BAA)), Bacillus sp. SG-1, Bacillus sp. 707, Bacillus sp. DSM12368 (i.e., A7-7), Bacillus sp. DSM 12649 (i.e., AA560), Bacillus sp. SP722, B. megaterium (DSM90 14), Cytophaga sp. (e.g., CspAmy2 amylase) and KSM AP1378.

The amino acid sequence of the mature form of engineered α-amylase 1 (VES33575M) is shown, below, as SEQ ID NO: 1:

AATNGTMMQYFEWYVPNDGQHWNKMKNDTAYLSSIGITALWIPPAYKGTS
QADVGYGAYDLYDLGEFNQKGTVRTKYGTKAELKSAINTLHSKGIQVYGD
VVMNHKAGADFTENVTAVEVNPSNRYQETSGEYNIQAWTGFNFPGRGTTY
SNWKWQWFHFDGTDWDQSRSLSRIFKFHGKAWDWPVSSENGNYDYLMYAD
YDYDHPDVVNEMKKWGVWYANEVGLDGYRLDAVKHIKESFLKDWVDNARA
ATGKEMFTVAEYWQNNLGEIENYLEKTGFNQSVEDVPLHYNFQAASSQGG
AYDMRNILNGTVTSKQPTRSVTFVDNHDTQPGQALESTVQSWFKPLAYAF
ILTREAGYPAVFYGDMYGTKGTSGYEIPSLKTKIEPLLKARKDYAYGTQR
DYIDNQDVIGWTREGDSTKAKSGLATVITDGPGGSKRMYVGKQNAGEVWY
DITGNRTDTVTINADGYGEFHVNGGSVSVYVQK

The amino acid sequence of the mature form of engineered α-amylase 2 (VES33367M) is shown, below, as SEQ ID NO: 2:

ASLNGTLMQYFEWYVPNDGQHWNRLQNDASYLSSVGITSLWIPPAYKGTS
QNDVGYGAYDLYDLGEFNQKGTVRTKYGTKAELKSAINTLHSKGIQVYGD
VVMNHKAGADATETVTAVEVNPNNRYQEISGEYQIQAWTGFNFPGRGNTY
SNWKWHWYHFDGVDWDQSRSLSRIYKFDGKAWDWPVSNEYGNYDYLMYAD
YDYDHPDVVNEMKKWGTWYANEVNLDGFRIDAAKHIKFSFLGDWVQSVRT
STGKEMFTVAEYWQNNLGSLENYLEKSGNNHSVEDVPLHYNFYAASTQSG
AYDMRNVLNGTVTAKYPTKSVTFVDNHDTQPGQSLESTVQTWFKPLAYAF
ILTREAGYPAVFYGDMYGTNGSTTYEIPALKSKIEPLLKARKDYAYGTQR
DYIDNPDVIGWTREGDPSVAASGLATVITDGPGGSKRMYVGRQHAGETWH
DITGNRSDPVTIHSDGYGEFHVNGGSVSIYVQK

The amino acid sequence of the mature form of engineered α-amylase 3 (VES33438M) is shown, below, as SEQ ID NO: 3:

ASTNGTMMQYFEWYVPNDGQHWNRLQNDASYLSSVGITALWIPPAYKGTS
QADVGYGAYDLYDLGEFNQKGTVRTKYGTKGELKSAINTLHSKGIQVYGD
VVMNHKAGADATEDVTAVEVNPNNRYQEISGEYQIEAWTGFDFPGRGNTY
SSFKWNWYHFDGVDWDQSRSLSRIYKFDGKAWDWPVSTEYGNYDYLMYAD
YDYDHPDVVNEMKKWGTWYANEVQLDGFRLDAVKHIKFSFLKDWVDNARA
ATGKEMFTVAEYWKNDLGALENYLEKTGFNQSVEDVPLHYNFHAASTQSG
AYDMRNVLNGTVTAKYPTKSVTFVENHDTQPGQSLESTVQSWFKPLAYAF
ILTRESGYPAVFYGDMYGTKGTTTYEIPALKSKIEPLLKARKDYAYGTQR
DYIDNQDVIGWTREGNTSKAKSGLATLITDGPGGSKRMYVGTQNAGEVWY
DITGNRTDTVTINADGYGEFAVNGGSVSVWVQK

The amino acid sequence of the mature form of engineered α-amylase 4 (VES35091M) is shown, below, as SEQ ID NO: 4:

ADNGTMMQYFEWYVPNDGQHWNKMKNDTAYLSSIGITAVWIPPAYKGTSQ
ADVGYGAYDLYDLGEFNQKGTVRTKYGTKAELKSAITTLHSKGIQVYGDV
VMNHKAGADFTENVTAVEVNPNNRYQEISGDYQIQAWTGFNFPGRGNTYS
SFKWNWFHFDGTDYDQSRNLNRIYKFTGKAWDWPVSTEYGNYDYLMYADY
DYDHPDVVNEMKKWGTWYANEVKLDGFRIDAAKHIKHSFLGDWVQSVRTS
TGKEMFTVAEYWQNNLGSLENYLEKSGNNHSVFDVPLHYNFQAASSQGGA
YDMRNILNGTVTSSQPTRSVTFVDNHDTQPGQALESTVQSWFKPLAYAFI
LTRESGYPAVFYGDMYGTKGTTGYEIPALKTKIEPLLKARKDFAYGTQRD
YIDNPDVIGWTREGNTSKANSGLATLITDGPGGAKRMYVGTQNAGEVWYD
LTGNRTDKVTIGSDGWATFNVNGGSVSVYVQQ

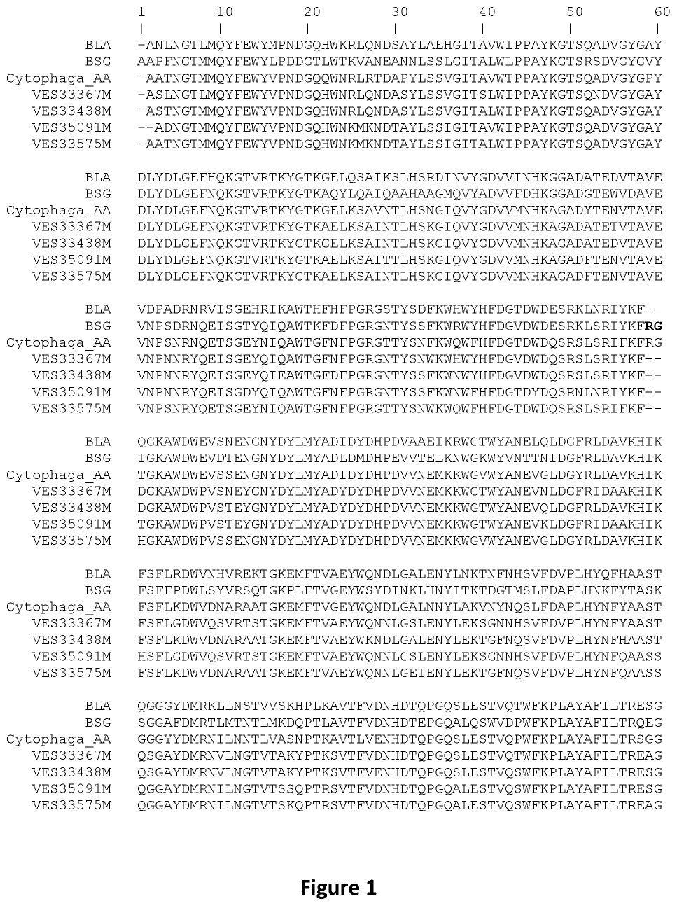

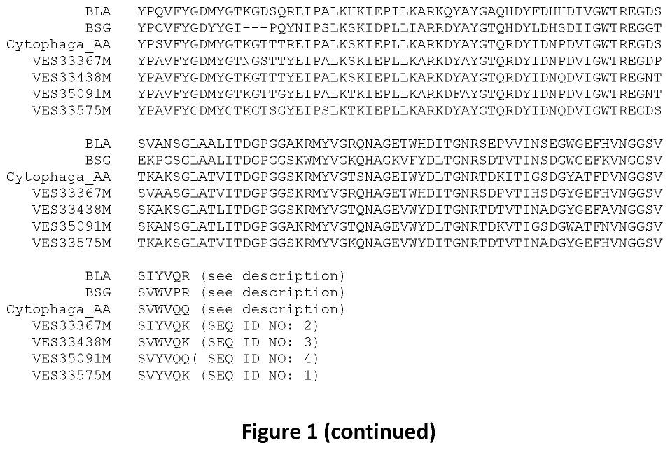

As shown in the amino acid sequence alignment in FIG. 1, all four engineered variants have a a deletion in the RG₁XG₂ motif adjacent to the calcium-binding loop corresponding to positions R179, G180, 1181 and G182 in the α-amylase from Bacillus stearothermophilus (SEQ ID NO: 16; shown in bold). This deletion is naturally present in the α-amylase from Bacillus lichenifomis (SEQ ID NO: 17). In the engineered α-amylases, this deletion is between residues F176 and K179 (referring to any of SEQ ID NO: 1-4). Note that it is well known that whether RG₁ or XG₂ in the motif is deleted makes no difference to performce and the resulting molecules are often difficult to distinguish based on subsequent amino acid sequence anaylsis.

In some embodiments, the engineered α-amylases are non-naturally-occuring and have at least 85%, at least 86%, at least 87%, at least 88%, at least 89%, at least 90%, at least 91%, at least 92%, at least 93%, at least 94%, at least 95%, at least 96%, at least 97%, at least 98% or even at least 99%, or more, amino acid sequence homology/identity to any of SEQ ID Nos: 1-4.

In some embodiments, the engineered α-amylases are non-naturally-occuring and have any number of conservative amino acid substitutions, which are well recognized in the art. The present engineered α-amylases may be “precursor,” “immature,” or “full-length,” in which case they include a signal sequence, or “mature,” in which case they lack a signal sequence. Mature forms of the polypeptides are generally the most useful. The present engineered α-amylases may also be truncated to remove the N or C-termini, or extended to include additional N or C-terminal residues, so long as the resulting polypeptides retains activity.

It is known that many bacterial (and other) α-amylases share the same fold, often share significant amino acid sequence identity, and sometimes benefit from the same mutations; therefore, the mutations described in other Family 13 α-amylases are expected to be transferable to the present engineered α-amylases.

### 4. Nucleotides Encoding Engineered α-Amylases

In another aspect, nucleic acids encoding an engineered α-amylase are provided. The nucleic acid may encode a particular engineered α-amylases, or an α-amylases having a specified degree of amino acid sequence identity to the particular engineered α-amylase.

In one example, the nucleic acid encodes an amylase at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% homology/identity to any of SEQ ID Nos: 5-8 (excluding the portion of the nucleic acid that encodes the signal sequence). It will be appreciated that due to the degeneracy of the genetic code, a plurality of nucleic acids may encode the same polypeptide.

In another example, the nucleic acid hybridizes under stringent or very stringent conditions to a nucleic acid encoding (or complementary to a nucleic acid encoding) an engineered α-amylases having at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% homology/identity to any of SEQ ID NOs: 1-4 In some embodiments, the nucleic acid hybridizes under stringent or very stringent conditions to the nucleic acid of any of SEQ ID NOs: 5-8, or to a nucleic acid complementary to these nucleic acids.

Nucleic acids may encode a “full-length” (“fl” or “FL”) amylase, which includes a signal sequence, only the mature form of an amylase, which lacks the signal sequence, or a truncated form of an amylase, which lacks the N or C-terminus of the mature form.

A nucleic acid that encodes a α-amylase can be operably linked to various promoters and regulators in a vector suitable for expressing the α-amylase in host cells. Exemplary promoters are from B. licheniformis amylase (LAT), B. subtilis (AmyE or AprE), and Streptomyces CelA. Such a nucleic acid can also be linked to other coding sequences, e.g., to encode a chimeric polypeptide.

### 5. Production of Engineered α-Amylases

The present engineered α-amylases can be produced in host cells, for example, by secretion or intracellular expression, using methods well-known in the art. Suitable assays can be used to monitor amylase activity in a sample, for example, by assays directly measuring reducing sugars such as glucose in the culture media. For example, glucose concentration may be determined using glucose reagent kit No. 15-UV (Sigma Chemical Co.) or an instrument, such as Technicon Autoanalyzer. α-amylase activity also may be measured by any known method, such as the PAHBAH or ABTS assays, described below.

Fermentation, separation, and concentration techniques are well known in the art and conventional methods can be used to prepare a concentrated, variant-α-amylase-polypeptide-containing solution. After fermentation, a fermentation broth is obtained, the microbial cells and various suspended solids, including residual raw fermentation materials, can be removed by conventional separation techniques in order to obtain an amylase solution. Filtration, centrifugation, microfiltration, rotary vacuum drum filtration, ultrafiltration, centrifugation followed by ultra-filtration, extraction, or chromatography, or the like, are generally used.

### 6. Compositions and Uses of Engineered α-Amylases

Engineered α-amylases are useful for a variety of industrial applications. For example, engineered α-amylases are useful in a starch conversion process, particularly in a saccharification process of a starch that has undergone liquefaction. The desired end-product may be any product that may be produced by the enzymatic conversion of the starch substrate. For example, the desired product may be a syrup rich in glucose and maltose, which can be used in other processes, such as the preparation of HFCS, or which can be converted into a number of other useful products, such as ascorbic acid intermediates (e.g., gluconate; 2-keto-L-gulonic acid; 5-keto-gluconate; and 2,5-diketogluconate); 1,3-propanediol; aromatic amino acids (e.g., tyrosine, phenylalanine and tryptophan); organic acids (e.g., lactate, pyruvate, succinate, isocitrate, and oxaloacetate); amino acids (e.g., serine and glycine); antibiotics; antimicrobials; enzymes; vitamins; and hormones.

The starch conversion process may be a precursor to, or simultaneous with, a fermentation process designed to produce alcohol for fuel or drinking (i.e., potable alcohol). One skilled in the art is aware of various fermentation conditions that may be used in the production of these end-products. Engineered α-amylases are also useful in compositions and methods of food preparation. These various uses of engineered α-amylases are described in more detail below.

### 6.1. Preparation of Starch Substrates

Those of general skill in the art are well aware of available methods that may be used to prepare starch substrates for use in the processes disclosed herein. For example, a useful starch substrate may be obtained from tubers, roots, stems, legumes, cereals or whole grain. More specifically, the granular starch may be obtained from corn, cobs, wheat, barley, rye, triticale, milo, sago, millet, cassava, tapioca, sorghum, rice, peas, bean, banana, or potatoes.

The starch from a grain may be ground or whole and includes corn solids, such as kernels, bran and/or cobs. The starch may also be highly refined raw starch or feedstock from starch refinery processes. Various starches also are commercially available.

The starch substrate can be a crude starch from milled whole grain, which contains non-starch fractions, e.g., germ residues and fibers. Milling may comprise either wet milling or dry milling or grinding. In wet milling, whole grain is soaked in water or dilute acid to separate the grain into its component parts, e.g., starch, protein, germ, oil, kernel fibers. Wet milling efficiently separates the germ and meal (i.e., starch granules and protein) and is especially suitable for production of syrups.

In dry milling or grinding, whole kernels are ground into a fine powder and often processed without fractionating the grain into its component parts. In some cases, oils and/or fiber from the kernels are recovered. Dry ground grain thus will comprise significant amounts of non-starch carbohydrate compounds, in addition to starch. Dry grinding of the starch substrate can be used for production of ethanol and other biochemicals.

### 6.2. Gelatinization and Liquefaction of Starch

Liquefaction refers to a process by which starch is converted to less viscous and shorter chain dextrins. Generally, this process involves gelatinization of starch simultaneously with or followed by the addition of an α-amylase, although additional liquefaction-inducing enzymes optionally may be added. The starch substrate is generally slurried with water. The starch slurry may contain starch as a weight percent of dry solids of about 10-55%, about 20-45%, about 30-45%, about 30-40%, or about 30-35%. The α-amylase typically used for this application is thermally stable. The α-amylase is usually supplied, for example, at about 1500 units per kg dry matter of starch. To optimize α-amylase stability and activity, the pH of the slurry typically is adjusted to about pH 4.5-6.5 and about 1 mM of calcium (about 40 ppm free calcium ions) can also be added, depending upon the properties of the amylase used. Bacterial α-amylase remaining in the slurry following liquefaction may be deactivated via a number of methods, including lowering the pH in a subsequent reaction step or by removing calcium from the slurry in cases where the enzyme is dependent upon calcium.

The slurry of starch plus the engineered α-amylase may be pumped continuously through a jet cooker, which is steam heated to 105° C. Gelatinization occurs rapidly under these conditions, and the enzymatic activity, combined with the significant shear forces, begins the hydrolysis of the starch substrate. The residence time in the jet cooker is brief. The partly gelatinized starch may be passed into a series of holding tubes maintained at 105-110° C. and held for 5-8 min. to complete the gelatinization process (“primary liquefaction”). Hydrolysis to the required DE is completed in holding tanks at 85-95° C. or higher temperatures for about 1 to 2 hours (“secondary liquefaction”). The slurry is then allowed to cool to room temperature. This cooling step can be 30 minutes to 180 minutes, e.g., 90 minutes to 120 minutes. The liquefied starch typically is in the form of a slurry having a dry solids content (w/w) of about 10-50%; about 10-45%; about 15-40%; about 20-40%; about 25-40%; or about 25-35%.

Liquefaction with engineered α-amylases advantageously can be conducted at low pH, eliminating the requirement to adjust the pH to about pH 4.5-6.5. Engineered α-amylases can be used for liquefaction at a pH range of 2 to 7, e.g., pH 3.0-7.5, pH 4.0-6.0, or pH 4.5-5.8. Variant amylases can maintain liquefying activity at a temperature range of about 85° C.-95° C., e.g., 85° C., 90° C., or 95° C. For example, liquefaction can be conducted with 800 μg an α-amylase in a solution of 25% DS corn starch for 10 min at pH 5.8 and 85° C., or pH 4.5 and 95° C., for example.

### 6.3. Saccharification

Liquefied starch can be saccharified into a syrup rich in lower DP (e.g., DP1+DP2) saccharides, using glucoamylases, optionally in the presence of another enzyme(s). Advantageously, the syrup obtainable using the provided variant amylases may contain a weight percent of DP2 of the total oligosaccharides in the saccharified starch exceeding 30%, e.g., 45%-65% or 55%-65%. The weight percent of (DP1+DP2) in the saccharified starch may exceed about 70%, e.g., 75%-85% or 80%-85%.

### 6.4. Isomerization

The soluble starch hydrolysate produced by treatment with amylase can be converted into high fructose starch-based syrup (HFSS), such as high fructose corn syrup (HFCS). This conversion can be achieved using a glucose isomerase, particularly a glucose isomerase immobilized on a solid support. The pH is increased to about 6.0 to about 8.0, e.g., pH 7.5 (depending on the isomerase), and Ca²⁺ is removed by ion exchange. Suitable isomerases include SWEETZYMER, IT (Novozymes A/S); G-ZYMER IMGI, and G-ZYMER G993, KETOMAXR, G-ZYMER G993, G-ZYMER G993 liquid, and GENSWEETR IGI. Following isomerization, the mixture typically contains about 40-45% fructose, e.g., 42% fructose.

### 6.5. Fermentation

The soluble starch hydrolysate, particularly a glucose rich syrup, can be fermented by contacting the starch hydrolysate with a fermenting organism (usually an ethanolagen) typically at a temperature around 32° C., such as from 30° C. to 35° C. for alcohol-producing yeast. The temperature and pH of the fermentation will depend upon the fermenting organism. EOF products include metabolites, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega 3 fatty acid, butanol, isoprene, 1,3-propanediol and other biomaterials.

Ethanologenic microorganisms include yeast, such as Saccharomyces cerevisiae and bacteria, e.g., Zymomonas moblis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. Microorganisms that produce other metabolites, such as citric acid and lactic acid, by fermentation are also known in the art.

The saccharification and fermentation processes may be carried out as an SSF process. Fermentation may comprise subsequent enrichment, purification, and recovery of ethanol, for example. During the fermentation, the ethanol content of the broth (or beer) may reach about 8-18% v/v, e.g., 14-15% v/v. The broth may be distilled to produce enriched, e.g., 96% pure, solutions of ethanol. CO² generated by fermentation may be collected with a CO2 scrubber, compressed, and marketed for other uses, e.g., carbonating beverage or dry ice production. Solid waste from the fermentation process may be used as protein-rich products, e.g., livestock feed.

A variation on this process is a “fed-batch fermentation” system, where the substrate is added in increments as the fermentation progresses. Fed-batch systems are useful when catabolite repression may inhibit the metabolism of the cells and where it is desirable to have limited amounts of substrate in the medium. The actual substrate concentration in fed-batch systems is estimated by the changes of measurable factors such as pH, dissolved oxygen and the partial pressure of waste gases, such as CO². Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density where cells are primarily in log phase growth. Continuous fermentation permits modulation of cell growth and/or product concentration. For example, a limiting nutrient such as the carbon source or nitrogen source is maintained at a fixed rate and all other parameters are allowed to be moderated. Because growth is maintained at a steady state, cell loss due to medium being drawn off should be balanced against the cell growth rate in the fermentation. Methods of optimizing continuous fermentation processes and maximizing the rate of product formation are well known in the art of industrial microbiology.

### 6.6. Compositions Comprising Engineered α-Amylases

Engineered α-amylases may be combined with a glucoamylase (EC 3.2.1.3), e.g., a Trichoderma glucoamylase or variant thereof. Alternatively, the glucoamylase may be another glucoamylase derived from plants (including algae), fungi, or bacteria

Other suitable enzymes that can be used with the engineered α-amylases include a phytase, protease, pullulanase, β-amylase, isoamylase, a different α-amylase, α-glucosidase, cellulase, xylanase, other hemicellulases, β-glucosidase, transferase, pectinase, lipase, cutinase, esterase, redox enzymes, or a combination thereof.

Compositions comprising the present amylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc, for example, endogenous plant (including algal) enzymes, residual enzymes from a prior processing step, and the like.

### 7. Compositions and Methods for Baking and Food Preparation

The present compositions and methods also relate to a food composition, including but not limited to a food product, animal feed and/or food/feed additives, comprising an amylase, and methods for preparing such a food composition comprising mixing engineered α-amylase with one or more food ingredients, or uses thereof.

Additionally, the present compositions and methods relate to the use of an engineered α-amylase in the preparation of a food composition, wherein the food composition is baked subsequent to the addition of the polypeptide of the invention.

An engineered α-amylase can further be added alone or in a combination with other amylases to prevent or retard staling, i.e., crumb firming of baked products. The amount of anti-staling amylase will typically be in the range of 0.01-10 mg of enzyme protein per kg of flour, e.g., 0.5 mg/kg ds. Additional anti-staling amylases that can be used in combination with an amylase include an endo-amylase, e.g., a bacterial endo-amylase from Bacillus.

The baking composition comprising an amylase further can comprise a phospholipase or enzyme with phospholipase activity. An enzyme with phospholipase activity has an activity that can be measured in Lipase Units (LU). The phospholipase may have A1 or A2 activity to remove fatty acid from the phospholipids, forming a lysophospholipid. It may or may not have lipase activity, i.e., activity on triglyceride substrates. The phospholipase typically has a temperature optimum in the range of 30-90° C., e.g., 30-70° C. The added phospholipases can be of animal origin, for example, from pancreas, e.g., bovine or porcine pancreas, snake venom or bee venom. Alternatively, the phospholipase may be of microbial origin, e.g., from filamentous fungi, yeast or bacteria, for example.

The phospholipase is added in an amount that improves the softness of the bread during the initial period after baking, particularly the first 24 hours. The amount of phospholipase will typically be in the range of 0.01-10 mg of enzyme protein per kg of flour, e.g., 0.1-5 mg/kg. That is, phospholipase activity generally will be in the range of 20-1000 LU/kg of flour, where a Lipase Unit is defined as the amount of enzyme required to release 1 μmol butyric acid per minute at 30° C., pH 7.0, with gum arabic as emulsifier and tributyrin as substrate.

Compositions of dough generally comprise wheat meal or wheat flour and/or other types of meal, flour or starch such as corn flour, cornstarch, rye meal, rye flour, oat flour, oatmeal, soy flour, sorghum meal, sorghum flour, potato meal, potato flour or potato starch. The dough may be fresh, frozen or par-baked. The dough can be a leavened dough or a dough to be subjected to leavening. The dough may be leavened in various ways, such as by adding chemical leavening agents, e.g., sodium bicarbonate or by adding a leaven, i.e., fermenting dough. Dough also may be leavened by adding a suitable yeast culture, such as a culture of Saccharomyces cerevisiae (baker's yeast), e.g., a commercially available strain of S. cerevisiae.

The dough may also comprise other conventional dough ingredients, e.g., proteins, such as milk powder, gluten, and soy; eggs (e.g., whole eggs, egg yolks or egg whites); an oxidant, such as ascorbic acid, potassium bromate, potassium iodate, azodicarbonamide (ADA) or ammonium persulfate; an amino acid such as L-cysteine; a sugar; or a salt, such as sodium chloride, calcium acetate, sodium sulfate or calcium sulfate. The dough further may comprise fat, e.g., triglyceride, such as granulated fat or shortening. The dough further may comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin. In particular, the dough can be made without addition of emulsifiers.

The dough product may be any processed dough product, including fried, deep fried, roasted, baked, steamed and boiled doughs, such as steamed bread and rice cakes. In one embodiment, the food product is a bakery product. Typical bakery (baked) products include bread-such as loaves, rolls, buns, bagels, pizza bases etc. pastry, pretzels, tortillas, cakes, cookies, biscuits, crackers etc.

Optionally, an additional enzyme may be used together with the anti-staling α-amylase and the phospholipase. The additional enzyme may be a second amylase, such as an amyloglucosidase, a β-amylase, a cyclodextrin glucanotransferase, or the additional enzyme may be a peptidase, in particular an exopeptidase, a transglutaminase, a lipase, a cellulase, a xylanase, a protease, a protein disulfide isomerase, e.g., a protein disulfide isomerase as disclosed in WO 95/00636, for example, a glycosyltransferase, a branching enzyme (1,4-α-glucan branching enzyme), a 4-α-glucanotransferase (dextrin glycosyltransferase) or an oxidoreductase, e.g., a peroxidase, a laccase, a glucose oxidase, an amadoriase, a metalloproteinase, a pyranose oxidase, a lipooxygenase, an L-amino acid oxidase or a carbohydrate oxidase. The additional enzyme(s) may be of any origin, including mammalian and plant, and particularly of microbial (bacterial, yeast or fungal) origin and may be obtained by techniques conventionally used in the art.

An engineered α-amylase may be used in a pre-mix, comprising flour together with an anti-staling amylase, a phospholipase, and/or a phospholipid. The pre-mix may contain other dough-improving and/or bread-improving additives, e.g., any of the additives, including enzymes, mentioned above. An amylase can be a component of an enzyme preparation comprising an anti-staling amylase and a phospholipase, for use as a baking additive.

### 8. Textile Desizing Compositions and Use, Thereof

Also contemplated are compositions and methods for treating fabrics (e.g., to desize a textile) using an engineered α-amylase. Fabric-treating methods are well known in the art. For example, the feel and appearance of a fabric can be improved by a method comprising contacting the fabric with an amylase in a solution. The fabric can be treated with the solution under pressure.

An engineered α-amylase can be applied during or after the weaving of a textile, or during the desizing stage, or one or more additional fabric processing steps. An engineered α-amylase can be applied during or after the weaving to remove these sizing starch or starch derivatives. After weaving, an amylase can be used to remove the size coating before further processing the fabric to ensure a homogeneous and wash-proof result.

An engineered α-amylase can be used alone or with other desizing chemical reagents and/or desizing enzymes to desize fabrics, including cotton-containing fabrics, as detergent additives, e.g., in aqueous compositions. An engineered α-amylase also can be used in compositions and methods for producing a stonewashed look on indigo-dyed denim fabric and garments.

### 9. Cleaning Compositions

An aspect of the present compositions and methods is a cleaning composition that includes an engineered α-amylase as a component. An engineered α-amylase polypeptide can be used as a component in detergent compositions for, e.g., hand washing, laundry washing, dishwashing, and other hard-surface cleaning. Such compositions include heavy duty liquid (HDL), heavy duty dry (HDD), and hand (manual) laundry detergent compositions, including unit dose format laundry detergent compositions, and automatic dishwashing (ADW) and hand (manual)dishwashing compositions, including unit dose format dishwashing compositions.

Preferably, an engineered α-amylase is incorporated into detergents at or near a concentration conventionally used for α-amylase in detergents. For example, an engineered α-amylase polypeptide may be added in amount corresponding to 0.00001-1 mg (calculated as pure enzyme protein) of amylase per liter of wash/dishwash liquor. Exemplary formulations are myriad in nature and the mere description (or claiming of novelty) of a known or slightly modified detergent formulations with the present engineered α-amylases should in no way be presumed to be inventive with genuine comparative data.

### 10. Brewing Compositions

The present engineered α-amylases may be a component of a brewing composition used in a process of brewing, i.e., making a fermented malt beverage. Non-fermentable carbohydrates form the majority of the dissolved solids in the final beer. This residue remains because of the inability of malt amylases to hydrolyze the α-1,6-linkages of the starch. The non-fermentable carbohydrates contribute about 50 calories per 12 ounces of beer. An engineered α-amylase, in combination with a glucoamylase and optionally a pullulanase and/or isoamylase, assist in converting the starch into dextrins and fermentable sugars, lowering the residual non-fermentable carbohydrates in the final beer.

All references cited herein are herein incorporated by reference in their entirety for all purposes. To further illustrate the compositions and methods, and advantages thereof, the following specific examples are given with the understanding that they are illustrative rather than limiting.

### EXAMPLES

### Example 1

### Construction of Strains Engineered α-Amylase Variants

Four engineered α-amylases were synthetically assembled. The identity of the engineered α-amylases and relevant amino acid and nucleic acid sequence information is summarized in Table 1. The engineered α-amylases numbers and internal reference numbers are used without distinction.

TABLE 1
Description of variants

| Engineered α- | | Protein SEQ ID | DNA SEQ ID |
|---|---|---|---|
| amylases no. | Internal ref. no. | NO | NO |
| 1 | VES33575M | 1 | 5 |
| 2 | VES33367M | 2 | 6 |
| 3 | VES33438M | 3 | 7 |
| 4 | VES35091M | 4 | 8 |

To express these α-amylases, DNA cassettes overexpressing engineered α-amylase 1, 2, 3 or 4 were each integrated into the cat locus of B. licheniformis strain BF62 (PCT Publication No. WO2018156705A1). The expression cassette contained a downstream homology arm to the cat gene (SEQ ID NO: 15), operably linked to the DNA encoding the Kanamycin resistance protein gene expression cassette (SEQ ID NO: 9), operably linked to the synthetic p3 promoter (SEQ ID NO: 10), operably linked to the DNA encoding the B. subtilis aprE 5′ UTR (SEQ ID NO: 11), operably linked to the DNA encoding the modified B. licheniformis amyL signal sequence (SEQ ID NO: 12), operably linked to the DNA encoding α-amylase 1, 2, 3 or 4 (SEQ ID NO: 5, 6, 7 or 8), operably linked to the B. licheniformis amyL transcriptional terminator (SEQ ID NO: 13), operably linked to the DNA encoding the upstream homology arm to the cat gene (SEQ ID NO: 14). DNA cassettes overexpressing engineered α-amylase 1, 2, 3 or 4 were constructed by making use of chemical DNA synthesis and/or overlap extension PCR techniques.

The four α-amylase overexpression DNA cassettes were each used to transform the BF62 strain using the method as described in WO2018156705A1. Briefly, the BF62 competent cells were generated by incubating BF62 cells in Luria broth containing 100 ppm spectinomycin at 37° C. with shaking. The cultures was diluted the next day to an OD₆₀₀ of 0.7 using fresh Luria broth again containing 100 ppm spectinomycin. The cultures were grown for one 1 hr at 37° C., with shaking at 250 RPM, and D-xylose was added to induce comK expression. The cultures were grown for an additional 4 hours at 37° C. with shaking at 250 RPM. Cells were harvested by centrifugations at 1700 g, and used as competent cells for transformation by making use of DNA cassettes of α-amylase 1, 2, 3, and 4. BF62 competent cells were mixed with an aliquot of the DNA cassettes. The cell/DNA mixtures were incubated at 37° C. for 1.5 hr with shaking at 1200 rpm, followed by plating on Heart Infusion (HI) agar plates containing 3 mg/L of neomycin trisulfate salt hydrate (Sigma-Aldrich, N1876-25G). The plates were incubated at 37° C. for 48 hr. Transformed colonies separately expressing each of the four engineered α-amylases were screened by PCR-amplification to confirm expected integration.

A colony expressing each of the α-amylases was cultured overnight in Luria broth supplemented with 5 mg/L neomycin trisulfate salt hydrate, and stored at −80° C. in 20% v/v glycerol.

To obtain sufficient amounts of the engineered α-amylase to assay for enzymatic performance, the cells were cultured using standard small scale or lab-scale fermentation conditions (see, e.g., PCT Publication Nos. WO2018/156705 and WO2019/055261).

The relevant amino acid and nucleic acid sequences are shown, below. The amino acid sequence of the mature form of engineered α-amylase 1 (VES33575M) is shown as SEQ ID NO: 1:

AATNGTMMQYFEWYVPNDGQHWNKMKNDTAYLSSIGITALWIPPAYKGTS
QADVGYGAYDLYDLGEFNQKGTVRTKYGTKAELKSAINTLHSKGIQVYGD
VVMNHKAGADFTENVTAVEVNPSNRYQETSGEYNIQAWTGFNFPGRGTTY
SNWKWQWFHFDGTDWDQSRSLSRIFKFHGKAWDWPVSSENGNYDYLMYAD
YDYDHPDVVNEMKKWGVWYANEVGLDGYRLDAVKHIKFSFLKDWVDNARA
ATGKEMFTVAEYWQNNLGEIENYLEKTGFNQSVEDVPLHYNFQAASSQGG
AYDMRNILNGTVTSKQPTRSVTFVDNHDTQPGQALESTVQSWFKPLAYAF
ILTREAGYPAVFYGDMYGTKGTSGYEIPSLKTKIEPLLKARKDYAYGTQR
DYIDNQDVIGWTREGDSTKAKSGLATVITDGPGGSKRMYVGKQNAGEVWY
DITGNRTDTVTINADGYGEFHVNGGSVSVYVQK

The amino acid sequence of the mature form of engineered α-amylase 2 (VES33367M) is shown, below, as SEQ ID NO: 2:

ASLNGTLMQYFEWYVPNDGQHWNRLQNDASYLSSVGITSLWIPPAYKGTS
QNDVGYGAYDLYDLGEFNQKGTVRTKYGTKAELKSAINTLHSKGIQVYGD
VVMNHKAGADATETVTAVEVNPNNRYQEISGEYQIQAWTGFNFPGRGNTY
SNWKWHWYHFDGVDWDQSRSLSRIYKFDGKAWDWPVSNEYGNYDYLMYAD
YDYDHPDVVNEMKKWGTWYANEVNLDGFRIDAAKHIKESFLGDWVQSVRT
STGKEMFTVAEYWQNNLGSLENYLEKSGNNHSVEDVPLHYNFYAASTQSG
AYDMRNVLNGTVTAKYPTKSVTFVDNHDTQPGQSLESTVQTWFKPLAYAF
ILTREAGYPAVFYGDMYGTNGSTTYEIPALKSKIEPLLKARKDYAYGTQR
DYIDNPDVIGWTREGDPSVAASGLATVITDGPGGSKRMYVGRQHAGETWH
DITGNRSDPVTIHSDGYGEFHVNGGSVSIYVQK

The amino acid sequence of the mature form of engineered α-amylase 3 (VES33438M) is shown, below, as SEQ ID NO: 3:

ASTNGTMMQYFEWYVPNDGQHWNRLQNDASYLSSVGITALWIPPAYKGTS
QADVGYGAYDLYDLGEFNQKGTVRTKYGTKGELKSAINTLHSKGIQVYGD
VVMNHKAGADATEDVTAVEVNPNNRYQEISGEYQIEAWTGFDFPGRGNTY
SSFKWNWYHFDGVDWDQSRSLSRIYKFDGKAWDWPVSTEYGNYDYLMYAD
YDYDHPDVVNEMKKWGTWYANEVQLDGFRLDAVKHIKESFLKDWVDNARA
ATGKEMFTVAEYWKNDLGALENYLEKTGFNQSVEDVPLHYNFHAASTQSG
AYDMRNVLNGTVTAKYPTKSVTFVENHDTQPGQSLESTVQSWFKPLAYAF
ILTRESGYPAVFYGDMYGTKGTTTYEIPALKSKIEPLLKARKDYAYGTQR
DYIDNQDVIGWTREGNTSKAKSGLATLITDGPGGSKRMYVGTQNAGEVWY
DITGNRTDTVTINADGYGEFAVNGGSVSVWVQK

The amino acid sequence of the mature form of engineered α-amylase 4 (VES35091M) is shown, below, as SEQ ID NO: 4:

ADNGTMMQYFEWYVPNDGQHWNKMKNDTAYLSSIGITAVWIPPAYKGTSQ
ADVGYGAYDLYDLGEFNQKGTVRTKYGTKAELKSAITTLHSKGIQVYGDV
VMNHKAGADFTENVTAVEVNPNNRYQEISGDYQIQAWTGFNFPGRGNTYS
SFKWNWFHFDGTDYDQSRNLNRIYKFTGKAWDWPVSTEYGNYDYLMYADY
DYDHPDVVNEMKKWGTWYANEVKLDGFRIDAAKHIKHSFLGDWVQSVRTS
TGKEMFTVAEYWQNNLGSLENYLEKSGNNHSVFDVPLHYNFQAASSQGGA
YDMRNILNGTVTSSQPTRSVTFVDNHDTQPGQALESTVQSWFKPLAYAFI
LTRESGYPAVFYGDMYGTKGTTGYEIPALKTKIEPLLKARKDFAYGTQRD
YIDNPDVIGWTREGNTSKANSGLATLITDGPGGAKRMYVGTQNAGEVWYD
LTGNRTDKVTIGSDGWATFNVNGGSVSVYVQQ

The nucleic acid sequence encoding the mature form of engineered α-amylase 1 is shown, below, as SEQ ID NO: 5:

GCAGCGACGAATGGAACGATGATGCAATATTTTGAATGGTATGTTCCAAATGATGGCCAGCATT
GGAACAAAATGAAGAATGATACGGCTTATTTATCAAGTATAGGGATCACTGCCCTTTGGATTCC
TCCGGCTTATAAAGGGACAAGCCAGGCGGATGTTGGCTACGGTGCATACGACCTTTATGACCTG
GGAGAATTTAATCAAAAAGGGACGGTTCGAACGAAATATGGAACAAAAGCTGAACTTAAATCTG
CCATCAATACTCTTCACAGCAAAGGCATTCAAGTATATGGCGATGTCGTAATGAATCATAAAGC
CGGAGCGGATTTTACTGAAAATGTAACAGCTGTGGAGGTCAATCCGTCAAACCGATACCAGGAA
ACATCCGGTGAATACAACATCCAAGCCTGGACGGGCTTTAACTTTCCAGGTAGAGGCACAACCT
ACTCCAACTGGAAATGGCAGTGGTTTCATTTCGACGGAACAGATTGGGATCAATCCAGATCACT
ATCAAGAATCTTTAAATTCCATGGAAAAGCATGGGATTGGCCAGTATCATCAGAAAACGGAAAC
TATGATTACTTAATGTATGCGGATTACGATTACGATCATCCGGATGTTGTAAACGAAATGAAAA
AGTGGGGAGTGTGGTATGCCAATGAAGTTGGCCTGGATGGATATAGGCTGGATGCTGTGAAACA
TATTAAGTTCTCCTTCCTTAAAGACTGGGTAGATAACGCGCGCGCGGCGACTGGAAAAGAAATG
TTTACAGTGGCAGAGTATTGGCAAAACAATCTTGGAGAAATTGAAAATTACTTAGAAAAAACAG
GCTTTAATCAGTCAGTATTTGATGTACCGCTCCACTATAACTTTCAGGCAGCCTCTTCACAAGG
CGGTGCCTATGATATGAGAAATATTTTAAATGGAACGGTTACTTCCAAACAGCCAACAAGATCG
GTAACGTTTGTAGATAATCATGATACACAGCCAGGACAGGCTCTGGAATCAACTGTGCAAAGCT
GGTTTAAACCTCTTGCTTATGCTTTCATATTGACACGGGAGGCGGGGTATCCAGCCGTGTTTTA
CGGGGATATGTACGGAACAAAAGGGACAAGCGGCTATGAAATTCCTAGCTTAAAAACAAAGATT
GAACCTTTATTAAAAGCGAGAAAAGACTACGCATACGGTACCCAGCGGGATTATATCGACAATC
AGGATGTCATAGGCTGGACAAGAGAAGGAGATTCCACAAAAGCCAAATCAGGACTGGCGACTGT
GATTACGGACGGTCCGGGAGGCTCAAAGCGGATGTATGTCGGTAAACAAAATGCAGGAGAAGTG
TGGTATGATATTACGGGGAATAGAACGGACACAGTAACTATAAACGCGGATGGCTATGGCGAAT
TTCATGTAAATGGCGGATCTGTATCCGTTTATGTCCAGAAATAA

The nucleic acid sequence encoding the mature form of engineered α-amylase 2 is shown, below, as SEQ ID NO: 6:

GCATCACTGAATGGAACGCTGATGCAATATTTTGAATGGTATGTTCCAAATGATGGCCAGCATT
GGAACAGACTGCAGAATGATGCGTCATATTTATCAAGTGTGGGGATCACTTCACTTTGGATTCC
TCCGGCTTATAAAGGGACAAGCCAGAACGATGTTGGCTACGGTGCATACGACCTTTATGACCTG
GGAGAATTTAATCAAAAAGGGACGGTTCGAACGAAATATGGAACAAAAGCTGAACTTAAATCTG
CCATCAATACTCTTCACAGCAAAGGCATTCAAGTATATGGCGATGTCGTAATGAATCATAAAGC
CGGAGCGGATGCGACTGAAACAGTAACAGCTGTGGAGGTCAATCCGAACAACCGATACCAGGAA
ATTTCCGGTGAATACCAAATCCAAGCCTGGACGGGCTTTAACTTTCCAGGTAGAGGCAATACCT
ACTCCAACTGGAAATGGCATTGGTATCATTTCGACGGAGTGGATTGGGATCAATCCAGATCACT
ATCAAGAATCTATAAATTCGATGGAAAAGCATGGGATTGGCCAGTATCAAACGAATACGGAAAC
TATGATTACTTAATGTATGCGGATTACGATTACGATCATCCGGATGTTGTAAACGAAATGAAAA
AGTGGGGAACCTGGTATGCCAATGAAGTTAACCTGGATGGATTCAGGATTGATGCTGCGAAACA
TATTAAGTTCTCCTTCCTTGGAGACTGGGTACAGTCAGTCCGCACCTCGACTGGAAAAGAAATG
TTTACAGTGGCAGAGTATTGGCAAAACAATCTTGGATCCCTTGAAAATTACTTAGAAAAATCCG
GCAATAATCACTCAGTATTTGATGTACCGCTCCACTATAACTTTTATGCAGCCTCTACACAATC
AGGTGCCTATGATATGAGAAATGTGTTAAATGGAACGGTTACTGCGAAATATCCAACAAAATCG
GTAACGTTTGTAGATAATCATGATACACAGCCAGGACAGTCACTGGAATCAACTGTGCAAACAT
GGTTTAAACCTCTTGCTTATGCTTTCATATTGACACGGGAGGCGGGGTATCCAGCCGTGTTTTA
CGGGGATATGTACGGAACAAACGGGTCAACAACATATGAAATTCCTGCGTTAAAATCAAAGATT
GAACCTTTATTAAAAGCGAGAAAAGACTACGCATACGGTACCCAGCGGGATTATATCGACAATC
CGGATGTCATCGGCTGGACAAGAGAAGGAGATCCGTCCGTGGCCGCGTCAGGACTGGCGACTGT
GATTACGGACGGTCCGGGAGGCTCAAAGCGGATGTATGTCGGTAGACAACATGCAGGAGAAACA
TGGCATGATATTACGGGGAATAGATCAGACCCGGTAACTATACATTCAGATGGCTATGGCGAAT
TTCATGTAAATGGCGGATCTGTATCCATTTATGTCCAGAAATAA

The nucleic acid sequence encoding the mature form of engineered α-amylase 3 is shown, below, as SEQ ID NO: 7:

GCATCAACGAATGGAACGATGATGCAATATTTTGAATGGTATGTTCCAAATGATGGCCAGCATT
GGAACAGACTGCAGAATGATGCGTCATATTTATCAAGTGTGGGGATCACTGCCCTTTGGATTCC
TCCGGCTTATAAAGGGACAAGCCAGGCGGATGTTGGCTACGGTGCGTACGACCTTTATGACCTG
GGAGAATTTAATCAAAAAGGGACGGTTCGAACGAAATATGGAACAAAAGGCGAACTTAAATCTG
CCATCAATACTCTTCACAGCAAAGGCATTCAAGTATATGGCGATGTCGTAATGAATCATAAAGC
CGGAGCGGATGCGACTGAAGATGTAACAGCTGTGGAGGTCAATCCGAACAACCGATACCAGGAA
ATTTCCGGTGAATACCAAATCGAAGCCTGGACGGGCTTTGACTTTCCAGGTAGAGGCAATACCT
ACTCCAGCTTTAAATGGAACTGGTATCATTTCGACGGAGTGGATTGGGATCAATCCAGATCACT
ATCAAGAATCTATAAATTCGATGGAAAAGCATGGGATTGGCCAGTATCAACCGAATACGGAAAC
TATGATTACTTAATGTATGCGGATTACGATTACGATCATCCGGATGTTGTAAACGAAATGAAAA
AGTGGGGAACCTGGTATGCCAATGAAGTTCAGCTGGATGGATTCAGGCTGGATGCTGTGAAACA
TATTAAGTTCTCCTTCCTTAAAGACTGGGTAGATAACGCGCGCGCGGCGACTGGAAAAGAAATG
TTTACAGTGGCAGAGTATTGGAAAAACGATCTTGGAGCGCTTGAAAATTACTTAGAAAAAACAG
GCTTTAATCAGTCAGTATTTGATGTACCGCTCCACTATAACTTTCATGCAGCCTCTACACAATC
AGGTGCCTATGATATGAGAAATGTGTTAAATGGAACGGTTACTGCGAAATATCCAACAAAATCG
GTAACGTTTGTAGAAAATCATGATACACAGCCAGGACAGTCACTGGAATCAACTGTGCAAAGCT
GGTTTAAACCTCTTGCTTATGCTTTCATATTGACACGGGAGTCTGGGTATCCAGCCGTGTTTTA
CGGGGATATGTACGGAACAAAAGGGACAACAACATATGAAATTCCTGCGTTAAAATCAAAGATT
GAACCTTTATTAAAAGCGAGAAAAGACTACGCATACGGTACCCAGCGGGATTATATCGACAATC
AGGATGTCATCGGCTGGACAAGAGAAGGAAATACATCCAAAGCCAAATCAGGACTGGCGACTCT
TATTACGGACGGTCCGGGAGGCTCAAAGCGGATGTATGTCGGTACACAAAATGCAGGAGAAGTG
TGGTATGATATTACGGGGAATAGAACGGACACAGTAACTATAAACGCGGATGGCTATGGCGAAT
TTGCGGTAAATGGCGGATCTGTATCCGTTTGGGTCCAGAAATAA

The nucleic acid sequence encoding the mature form of engineered α-amylase 4 is shown, below, as SEQ ID NO: 8:

GCAGATAATGGAACGATGATGCAATATTTTGAATGGTATGTTCCAAATGATGGCCAGCATTGGA
ACAAAATGAAGAATGATACGGCTTATTTATCAAGTATAGGGATCACTGCCGTTTGGATTCCTCC
GGCTTATAAAGGGACAAGCCAGGCGGATGTTGGCTACGGTGCATACGACCTTTATGACCTGGGA
GAATTTAATCAAAAAGGGACGGTTCGAACGAAATATGGAACAAAAGCTGAACTTAAATCTGCCA
TTACCACACTTCACAGCAAAGGCATTCAAGTATATGGCGATGTCGTAATGAATCATAAAGCAGG
AGCGGATTTTACTGAAAATGTAACAGCTGTGGAGGTCAATCCGAACAACCGATACCAGGAAATT
TCCGGTGATTACCAAATCCAAGCCTGGACGGGCTTTAACTTTCCAGGTAGAGGCAATACCTACT
CCAGCTTTAAATGGAACTGGTTTCATTTCGACGGAACAGATTATGATCAATCCAGAAATCTAAA
CAGAATCTATAAATTCACCGGAAAAGCATGGGATTGGCCAGTATCAACCGAATACGGAAACTAT
GATTACTTAATGTATGCGGATTACGATTACGATCATCCGGATGTTGTAAACGAAATGAAAAAGT
GGGGAACCTGGTATGCCAATGAAGTTAAGCTGGATGGATTCAGGATTGATGCTGCGAAACATAT
TAAGCATTCCTTCCTTGGAGACTGGGTACAGTCAGTCCGCACCTCGACTGGAAAAGAAATGTTT
ACAGTGGCAGAGTATTGGCAAAACAATCTTGGATCCCTTGAAAATTACTTAGAAAAATCCGGCA
ATAATCACTCAGTATTTGATGTACCGCTCCACTATAACTTTCAGGCAGCCTCTTCACAAGGCGG
TGCCTATGATATGAGAAATATTTTAAATGGAACGGTTACTTCCTCACAGCCAACAAGATCGGTA
ACGTTTGTAGATAATCATGATACACAGCCAGGACAGGCTCTGGAATCAACTGTGCAAAGCTGGT
TTAAACCTCTTGCTTATGCTTTCATATTGACACGGGAGTCTGGGTATCCAGCCGTGTTTTACGG
GGATATGTACGGAACAAAAGGGACAACAGGCTATGAAATTCCTGCGTTAAAAACAAAGATTGAA
CCTTTATTAAAAGCGAGAAAAGACTTTGCATACGGTACCCAGCGGGATTATATCGACAATCCGG
ATGTTATCGGCTGGACAAGAGAAGGAAATACTTCCAAAGCCAATTCAGGACTGGCGACTCTTAT
TACGGACGGTCCGGGAGGCGCTAAGCGGATGTATGTCGGTACACAAAATGCAGGAGAAGTTTGG
TATGATCTAACGGGGAATAGAACGGACAAAGTAACTATAGGTTCAGATGGCTGGGCGACATTTA
ATGTAAATGGCGGATCTGTATCCGTTTATGTCCAGCAGTAG

The nucleic acid sequence of the kanamycin resistance protein gene expression cassette; coding sequence underlined (SEQ ID NO: 9)

ATCGGCTCCGTCGATACTATGTTATACGCCAACTTTCAAAACAACTTTGAAAAAGCTGTTTTCT
GGTATTTAAGGTTTTAGAATGCAAGGAACAGTGAATTGGAGTTCGTCTTGTTATAATTAGCTTC
TTGGGGTATCTTTAAATACTGTAGAAAAGAGGAAGGAAATAATAAATGGCTAAAATGAGAATAT
CACCGGAATTGAAAAAACTGATCGAAAAATACCGCTGCGTAAAAGATACGGAAGGAATGTCTCC
TGCTAAGGTATATAAGCTGGTGGGAGAAAATGAAAACCTATATTTAAAAATGACGGACAGCCGG
TATAAAGGGACCACCTATGATGTGGAACGGGAAAAGGACATGATGCTATGGCTGGAAGGAAAGC
TGCCTGTTCCAAAGGTCCTGCACTTTGAACGGCATGATGGCTGGAGCAATCTGCTCATGAGTGA
GGCCGATGGCGTCCTTTGCTCGGAAGAGTATGAAGATGAACAAAGCCCTGAAAAGATTATCGAG
CTGTATGCGGAGTGCATCAGGCTCTTTCACTCCATCGACATATCGGATTGTCCCTATACGAATA
GCTTAGACAGCCGCTTAGCCGAATTGGATTACTTACTGAATAACGATCTGGCCGATGTGGATTG
CGAAAACTGGGAAGAAGACACTCCATTTAAAGATCCGCGCGAGCTGTATGATTTTTTAAAGACG
GAAAAGCCCGAAGAGGAACTTGTCTTTTCCCACGGCGACCTGGGAGACAGCAACATCTTTGTGA
AAGATGGCAAAGTAAGTGGCTTTATTGATCTTGGGAGAAGCGGCAGGGCGGACAAGTGGTATGA
CATTGCCTTCTGCGTCCGGTCGATCAGGGAGGATATCGGGGAAGAACAGTATGTCGAGCTATTT
TTTGACTTACTGGGGATCAAGCCTGATTGGGAGAAAATAAAATATTATATTTTACTGGATGAAT
TGTTTTAGTGACTGCAGTGAGATCTGGTAATGACTCTCTAGCTTGAGGCATCAAATAAAACGAA
AGGCTCAGTCGAAAGACTGGGCCTCGAG

The nucleic acid sequence of the synthetic p3 promoter is shown, below, as SEQ ID NO: 10:

GTCGCTGATAAACAGCTGACATCAATATCCTATTTTTTCAAAAAATATT
TTAAAAAGTTGTTGACTTAAAAGAAGCTAAATGTTATAGTAATAAA

The nucleic acid sequence of the B. subtilis aprE 5′-UTR region is shown, below, as SEQ ID NO: 11:

ACAGAATAGTCTTTTAAGTAAGTCTACTCTGAATTTTTTTAAAAGGAGA
GGGTAAAGA

The nucleic acid sequence encoding the modified B. licheniformis amyL signal peptide coding sequence is shown, below, as SEQ ID NO: 12:

ATGAAACAACAAAAACGGCTTTACGCCCGATTGCTGACGCTGTTATTTG
CGCTCATCTTCTTGCTGCCTCATTCTGCAGCTTCAGCA

The nucleic acid sequence of the B. licheniformis amyL transcriptional terminator is shown, below, as SEQ ID NO: 13:

AAGAGCAGAGAGGACGGATTTCCTGAAGGAAATCCGTTTTTTTATTTT

The nucleic acid sequence of the upstream homology arm of the native B. licheniformis cat gene is shown, below, as SEQ ID NO: 14:

TAACATCTCTCACTGCTGTGTGATTTTACTCACGGCATTTGGAACGCCGGCTCTCAACAAACTT
TCTGTAGTGAAAATCATGAACCAAACGGATCGTCGGCCTGATTAACAGCTGAAAGCTGCCGATC
ACAAACATCCATAGTCCCGCCGGCTTCAGTTCCTCGGAGAAAAAGCAGAAGCTCCCGACAAGGA
ATAAAAGGCCGATGAGAAAATCGTTTAATGTATGTAGAACTTTGTATCTTTTTTTGAAAAAGAG
TTCATATCGATTGTTATTGTTTTGCGGCATTGCTTGATCACTCCAATCCTTTTATTTACCCTGC
CGGAAGCCGGAGTGAAACGCCGGTATACATAGGATTTATGAATTAGGAAAACATATGGGGAAAT
AAACCATCCAGGAGTGAAAAATATGCGGTTATTCATATGTGCATCGTGCCTGTTCGGCTTGATT
GTTCCGTCATTTGAAACGAAAGCGCTGACGTTTGAAGAATTGCCGGTTAAACAAGCTTCAAAAC
AATGGGAAGTTCAAATCGGTAAAGCCGAAGCCGGAAACGGAATGGCGAAACCGGAAAAAGGAGC
GTTTCATACTTATGCTGTCGAAATCAAAAACATTGGACACGATGTGGCTTCGGCGGAAATTTTT
GTCTATCGGAACGAGCCTAATTCTTCAACGAAATTTTCGCTTTGGAACATTCCTCACGAAAATC
CGGTTTCTTTAGCCAAAAGCTTAAATCACGGAAGCTCTGTCAAGCACCGCAATCTGCTTATGGC
AGAGAATGCGACCGAATTGGAAGTGGACATGATTTGGACGGAAAAAGGAAGCGAAGGCAGACTT
TTAAAGGAAACGTTCATTTTCAAGGGAGATGAATCATGAAGAAAAAATGGCCGTTCATCGTCAA
CGGTCTTTTTTTAATGACTTAGGCAGCCGATCGTTCGGCCATACGATATCGAAGCGACCTCGAA
CCAGCAGAGCTCGTCACAAAACATTTGCATTTAAAGAAAAATACAGGATGTTTTCACCAATATT
TTTCTCAATGATGATACACTATTGACAAGCTGCTACTTTGGGAGGGTGTTTCCATAGATGCCGA
TGAAGCAAAAACACCAAATGTGTCATGAGAGCTCTCTCTAATCGATATAAAAGTAGGGTGAACC
GGGGTTGTCAATCTGTAAAAGATCTTTTTTTATCCCGTGATACGCTTTTGGAATTCTGAATCTT
CAAGAAAGTCCCCAGCCTTTTGCTGATCAATCGAGAACAAAGGATGATACATATGAAAAGAATA
GATAAAATCTACCATCAGCTGCTGGATAATTTTCGCGAAAAGAATATCAATCAGCTTTTAAAGA
TACAAGGGAATTCGGCTAAAGAAATCGCCGGGCAGCTGCAAATGGAGCGTTCCAATGTCAGCTT
TGAATTAAACAATCTGGTTCGGGCCAAAAAGGTGATCAAGATTAAAACGTTCCCCGTCCGCTAC
ATCCCGGTGGAAATTGTTGAAAACGTCTTGAACATCAAATGGAATTCAGAGTTGATGGAGGTTG
AAGAACTGAGGCGGCTGGCTGACGGCCAAAAAAAGCCGGCGCGCAATATATCCGCCGATCCCCT
CGAGCTCATGATCGGGGCTAAAGGGAGCTTGAAAAAGGCAATTTCTCAGGCGAAAGCGGCAGTC
TTTTATCCTCCGCACGGCTTGCATATGCTGCTGCTCGGGCCGACGGGTTCGGGGAAATCGCTGT
TTGCGAATCGGATCTACCAGTTCGCCGTTTATTCTGACATATTGAAGCCCGATTCCCCGTTCAT
CACATTCAACTGTGCAGATTACTATAACAACCCTCAATTATTGCTCTCTCAATTGTTCGGACAT
AAAAAAGGGTCTTTTACAGGTGCGGGTGAAGACAAAGCAGGATTAGTCGAGCAGGCGGACGGGG
GCATTCTGTTTATGGATGAAATCCATCGCCTCCCGCCGGAGGGGCAGGAAATGCTGTTTTATTT
CATAGACAGCGGCACATACAACAGGCTTGGTGAAACAGAGCATAAACGAACGGCAAAAGTCCTG
TTTATCTGTGCGACAACAG

The nucleic acid sequence of the downstream homology arm of the native B. licheniformis cat gene is shown, below, as SEQ ID NO: 15:

CGATTAAACACGGCTACCGCAGTATTGATACCGCAGCCATCTACGGTAATGAAGAGGGGGTTGG
GCAAGGAATCCGCGAGGGGTTGAAAGAAGCCGGCATTTCAAGAGAAGACCTGTTTGTTACATCA
AAGGTCTGGAATGACGATTTAGGCTATGACGAAACGATTGCAGCCTATGAGGCGAGTCTCGAAA
AGCTCGGACTTGACTACCTTGATTTATACCTGATCCACTGGCCTGTTGAAGGACGCTACAAAGC
GGCGTGGAAAGCGCTTGAAACACTTTATGAACAAGGACGCGTAAAAGCAATCGGAGTGAGCAAT
TTTCAGATTCACCATCTGGAAGACTTGCTGAAAGATGCCGCCGTCAAACCGGCGATCAACCAGG
TTGAGTATCATCCGCGGCTGACGCAGAAAGAGCTGCAAGCGTTTTGCCGTGCGCACGGCATCCA
GCTGCAAGCATGGTCGCCGCTGATGCAAGGCCAATTGCTCAGCCATCCACTGCTGAAAGATATC
GCGGACAAGTACGGCAAGACACCGGCCCAAGTCATTTTGCGCTGGGATTTGCAAAACGGGGTCG
TTACGATTCCGAAGTCGACTAAAGCGGAGCGGATTGCCCAAAACGCGGACATATTTGATTTTGA
ACTGACCACCGAGGAAATGAAGCAAATTGACGCGCTGAATGAAAACACCCGTGTCGGCCCTGAT
CCCGATAACTTTGACTTTTAACAAAACGGCCCCGTTCGACATTCGAACGGGGCTTTAATTGAAT
TGTGCGGTTACACCGCCGGACTCCATCATCATCAGTTCTTTTTTCATATCCAATCCGCCCCGGT
ATCCCGTGAGCTGCCCGCTTTTACCGATAACCCGATGGCAAGGCACCACCATTAACAGCGGATT
TGCGCCGATCGCCGCGCCTACTGCCCGCACAGCGGCCTGCTTTTCAATATGCTCGGCGATATCG
GAATAGGAGCAAGTGCTGCCGTAAGGGATTTCGGAGAGCGCCTTCCACACTGCCAGCTGAAAAG
GCGTGCCGGCAAGGTCGACAGGAAAGCTGAAATGAGTTCGCTTGCCGTTCAAATACGCCTGCAG
CTGCTCGGCGTATTCTGCCAATCCTTTGTCATCCCGAATGAAAACTGGCTGTGTAAATCTTTTT
TCAGCCCAAGCGGCCAAATCCTCGAAGCCTTGATTCCATCCCCCTGTAAAACAGAGCCCGCGGG
CAGTCGCCCCAATGTGAATCTGCCAACCTCGGCAAATAAGCGTACGCCAGTATACGATTTGATC
GTCCATATGTTTACCTCCGTTTCATTTGCCGGTACGACGTCGGCGATTGCCCAGTCTTCTTTTT
AAACAAAGAGGCAAAATATTCCGCATTCGCAATGCCTACCATTGAAGCGATTTCTGCGATCGAT
CGTTCTGAATGAGCAAGCAAATCGACCGCTTTCTCAATCCTTTTCTGCAGGATGTATTCTGCCG
GCGAGACGCCTTTGATTCGTTTAAATGTCCGCTGCAGGTGAAAAGGGCTGATATGGCACCTGTC
AGCCAAAGCTTGCAGAGACAGCGGATCGCGATAAGATTCCTCGATGATTTCCACCACACGCTGT
GCCAGCTCTTCATCCGGCAGCAGCGCCCCGGCCGGATTGCAGCGTTTGCAGGGGCGGTACCCTT
CTGATAAAGCATCTTTTGCATTGAAAAAGATCTGCACATTGTCGATTTGCGGAACTCTCGATTT
GCAGGAAGGGCGGCAAAATATGCCGGTCGTTTTGACCGCGTAATAAAAAACTCCGTCATAGGCG
GAATCGTTTTCCGTAATCGCCCGCCACATTTCAGGCGTCAATCGTGATTTGCTGTTCATATCTT
CACCCCGATCTATGTCAGTATAACCTATATGACAGCCGGAGGTGGAGAGGCGGAGAACGGCACA
GCAAGAAGACAAAGAAGAAGAGAGACTGTTGCCTGGACCTCCGAAACGCGCTACAATTCATTTA
CAACACAGGATGGGGTGAGAATATTGCCGGAATCAGTGAAGCAGGCCTCCTAAA

### Example 2

### Laboratory Scale Liquefaction Assay

Liquefaction performance of the four engineered α-amylases cloned and expressed in Example 1 was evaluated using a laboratory scale corn starch liquefaction assay. Briefly, 35% dry solid of corn starch slurry was prepared by mixing together 9.75 g of corn starch (Ingredion BUFFALO 034010-102) and 15.25 g of Milli-Q water in a sample canister. The pH was adjusted to a preselected value using a 1 M potassium hydroxide or sulfuric acid solution. α-amylase was then added and mixed. Incubation was performed in a rapid viscosity analyzer (Perten RVA 4800) at 90° C., ramped to 110° C. over 2 min, held at 110° C. for 7 min, and then cooled to 95° C. over 1 min. The reaction mixtures were transferred into 50 ml conical tubes and further incubated in a water bath (Thermofisher) at 95° C. for 2 hr. An portion of each reaction mixture was diluted 200-fold in a 20 mM sulfuric acid solution and used for dextrose equivalents (DE) measurement.

The DE of liquefact was determined by measuring the quantity of reducing sugars (as glucose equivalent) using the BCA assay kit (Generay). 100 μL of BCA working solution and 5 μL of each 200-fold-diluted sample weas mixed in a PCR microplate (Axygen), which was incubated in a Thermo Cycler (T100, Bio-Rad) at 95° C. for 3 min, then cooled down to 20° C. 80 μL of each sample was then transferred to a new microplate (Costar 9017) and the absorbance was read at 562 nm. The amount of reducing sugar in the sample was determined by comparison to a known glucose standard. The percentage of glucose equivalent to the total carbohydrate (w/w) in the sample was then calculated as DE. The control enzymes used in all Examples are described in Table 2.

TABLE 2
Description of control enzymes

| Enzyme | Description |
|---|---|
| SPEZYME ® HT ™ | Variant of the α-amylase from a Cytophaga sp. (C16E in |
| | WO2014164777) |
| SPEZYME ® ALPHA ™ | Variant of the α-amylase from Bacillus stearothermophilus |
| | with the mutation S242Q (WO2009061381) |
| SPEZYME ® CL ™ | Variant of the α-amylase from Bacillus stearothermophilus |
| | with the mutation E188P (WO2009149130) |
| SPEZYME ® FRED ™ | Varinat of the α-amylase from Bacillus lichenifomis |
| | (WO9639528) |
| SPEZYMER SL ™ | Variant of the α-amylase from a Cytophaga sp. (C16F in |
| | WO2014164777) |
| LPHERA ® | WO2019113415 (SEQ ID NO: 16); WO2016087445 (SEQ |
| | ID NO: 25) |

The liquefaction performance of the α-amylases under low pH (i.e., 4.5 and 4.8) are summarized in Tables 3 and 4, respectively. Among all tested samples, VES33367M showed the best performance at pH 4.5 at equal enzyme dose. At pH 4.8, VES33367M, VES33438M, VES35091M and VES33575 all produced acceptable DE results.

Table 5 lists the liquefaction performance at pH higher than 5.0. VES33367M and VES33438M showed stable performance from pH 5.0 to pH 5.8 with low DE fluctuation under low enzyme dose. For some samples, DE was not determined (nd) due to high viscosity.

TABLE 3
Liquefaction performance at pH 4.5 at a dose of
2.85 μg enzyme/gds without extra ion addition

| Sample | DE |
|---|---|
| VES33367M | 12.7 |
| VES33438M | 10.2 |
| VES35091M | 8.4 |
| VES33575 | nd |
| SPEZYME ® SL ™ | nd |
| SPEZYME ® HT ™ | nd |
| SPEZYME ® ALPHA ™ | nd |
| SPEZYME ® CL ™ | nd |
| SPEZYME ® FRED ™ | nd |
| LPHERA ® | 6.8 |

TABLE 4
Liquefaction performance at pH 4.8 at a dose of
2.85 μg enzyme/gds without extra ion addition

| Sample | DE |
|---|---|
| VES33367M | 14.7 |
| VES33438M | 13.4 |
| VES35091M | 15.0 |
| VES33575 | 10.3 |
| SPEZYME ® SL ™ | nd |
| SPEZYME ® HT ™ | nd |
| SPEZYME ® ALPHA ™ | nd |
| SPEZYME ® CL ™ | nd |
| SPEZYME ® FRED ™ | nd |
| LPHERA ® | 11.0 |

TABLE 5
Liquefaction performance pH 5.0 or above

| | | Dosage | | Extra ions |
|---|---|---|---|---|
| Sample | pH | (μg) | DE | addition |
| VES33367M | 5.0 | 1.425 | 12.3 | No |
| | 5.3 | | 12.3 | |
| | 5.5 | | 12.5 | |
| | 5.8 | | 13.3 | |
| VES33438M | 5.0 | 1.425 | 11.4 | No |
| | 5.3 | | 13.4 | |
| | 5.5 | | 13.3 | |
| | 5.8 | | 14.1 | |
| VES35091M | 5.0 | 1.71 | 12.5 | No |
| | 5.3 | | 13.9 | |
| | 5.5 | | 14.0 | |
| | 5.8 | | 15.3 | |
| VES33575 | 5.0 | 2.85 | 12.8 | No |
| | 5.3 | 1.71 | 12.9 | |
| | 5.5 | | 13.5 | |
| | 5.8 | | 15.1 | |
| SPEZYME ® SL ™ | 5.5 | 4.275 | 9.7 | No |
| | 5.8 | 5.7 | 13.8 | 50 ppm Ca²⁺; |
| SPEZYME ® HT ™ | 5.8 | 2.85 | 14.0 | 100 ppm Na⁺ |
| SPEZYME ® ALPHA ™ | 5.8 | 5.7 | 14.9 | |
| SPEZYME ® CL ™ | 5.8 | 5.7 | 14.9 | |
| SPEZYME ® FRED ™ | 5.8 | 14.25 | 10.5 | |
| LPHERA ® | 5.0 | 2.85 | 11.8 | No |
| | 5.3 | | 12.1 | |
| | 5.5 | | 12.4 | |
| | 5.8 | | 13.0 | |

### Example 3

### Laboratory Scale Saccharification Assays

Liquefacts from Example 2 with DE values from 10 to 14 were selected for evaluation in a saccharification assay. Prior to the saccharification, the liquefacts were adjusted to a pH<3 and heated at 95° C. for 30 min, then adjusted back to pH 4.5. For the saccharification assay, 95 μL of each liquefact was transferred to a new microplate (Corning 3357), and 0.16 GAU/gds of glucoamylase (OPTIMAX® 4060, Danisco US Inc.) was added to initiate the saccharification reactions. The plate was incubated in an iEMS shaking incubator (Thermofisher) at 60° C. for 48 hours. At the end of incubation, the reaction mixtures were diluted 40-fold in 5 mM sulfuric acid. The DP composition was analyzed by HPLC using an ROA-Fast acid H+ column at 80° C. and an RID detector. 5 mM sulfuric acid solution was used as mobile phase at a flow rate of 1 mL/min. The results are summarized in Table 6.

TABLE 6
DP profiles of selected selected liquefacts
following addition of glucoamylase

| | | DP1 | DP2 | DP3 | DP3+ |
|---|---|---|---|---|---|
| Sample | DE | (%) | (%) | (%) | (%) |
| VES33367M | 12.9 | 95.6 | 2.0 | 0.7 | 1.8 |
| VES33438M | 10.6 | 95.5 | 1.9 | 0.6 | 2.0 |
| VES35091M | 13.4 | 95.6 | 2.0 | 0.7 | 1.8 |
| VES33575 | 12.1 | 95.6 | 2.0 | 0.7 | 1.8 |
| SPEZYME ® SL ™ | 10.7 | 95.5 | 2.0 | 0.6 | 1.8 |
| SPEZYME ® HT ™ | 12.1 | 95.4 | 2.0 | 0.7 | 1.9 |
| SPEZYME ® ALPHA ™ | 13.1 | 95.4 | 1.9 | 0.7 | 1.9 |
| SPEZYME ® CL ™ | 11.4 | 95.5 | 2.0 | 0.6 | 1.9 |
| LPHERA ® | 12.5 | 95.5 | 1.9 | 0.6 | 2.0 |

### Example 4

### Specific Activity Assays

1% (w/v) corn starch (Ingredion Inc.) was dissolved in 50 mM potassium acetate buffer at pH 4.5 with 5 ppm Ca²⁺ and 20 ppm Na⁺. Dissolved corn starch was boiled in a microwave oven, then cooled to room temperature overnight with gentle stirring. Each of the four engineered α-amylase-molecules (i.e., VES33367M, VES33575, VES33438M VES35091M), along with SPEZYMER HT™ and SPEZYME® SL™ were diluted to 0.2 ppm in 20 mM potassium acetate buffer at pH4.5 with 5 ppm Ca²⁺, and 20 ppm Na⁺ with 0.002% TWEEN80® (Sigma-Aldrich). 90 μL of 1% substrate was mixed with 9 μL of 0.2 ppm enzyme in a 96-well PCR microtiter plate and incubated in a thermoblock at 95° C. for 30 minutes. The reactions were cooled to 25° C. following incubation.

Alpha amylases activity was measured by detecting the reducing sugar equivalents generated in starch assays using Pierce BCA Protein Assay Kit (ThermoFisher, 23224). After the reactions were cooled to room temperature, 10 μL of reaction mixture was added to 90 μL BCA reagent in a 96-well PCR microtiter plate. This mixture was heated to 95° C. for 3 minutes. 80 μL of heated BCA reaction was then transferred to a polystyrene read plate and absorbance was measured at 562 nm.

Table 7 shows the relative enzymatic activities of the selected molecules compared to SPEZYME® HT™ and SPEZYME® SL™ benchmark amylases, where the activities of benchmark molecules were set to 100%. Activities at high temperatures were significantly greater for all of four engineered α-amylases compared to both benchmark α-amylases.

TABLE 7
Relative enzyme activity at pH 4.5, 95° C. for
30′, compared to benchmark α-amylases

| | % Relative to | % Relative to |
|---|---|---|
| Samples | SPEZYME ® HT ™ | SPEZYME ® SL ™ |
| VES33367M | 418 | 369 |
| VES33575 | 326 | 288 |
| VES33438M | 675 | 595 |
| VES35091M | 354 | 312 |
| SPEZYME ® HT ™ | (100) | 88 |
| SPEZYME ® SL ™ | 113 | (100) |

### Example 5

### Measurement of Thermostability

1% (w/v) corn starch (Ingredion Inc.) was dissolved in 50 mM potassium acetate buffer at pH5.6 with 80 ppm Ca²⁺ and 320 ppm Na⁺. Dissolved corn starch was boiled in a microwave, then cooled to room temperature overnight with gentle stirring. Each of the four engineered α-amylase-molecules (i.e., VES33367M, VES33575, VES33438M VES35091M), along with SPEZYME® HT™ and SPEZYME® SL™ were diluted to 0.04 ppm in 50 mM potassium acetate buffer at pH4.5 with 5 ppm Ca²⁺ and 20 ppm Na⁺ with 0.002% TWEEN80® (Sigma-Aldrich). 90 μL of 1% substrate was mixed with 9 μL of 0.04 ppm unstressed enzyme in a 96-well microtiter plate and incubated in an iEMS shaking incubator (Thermo Scientific) for 30 min at 60° C. The reactions were cooled down to 25° C. following incubation.

50 μL of 0.04 ppm enzyme dilutions were incubated in a 96-well PCR microtiter plate and incubated in a thermoblock at 94° C. for 10 min. The microtiter plate was cooled down to 25° C. following incubation. 90 μL of 1% substrate was mixed with 9 μL of 0.04 ppm heat stressed enzyme in a 96-well microtiter plate and incubated in an iEMS shaking incubator (Thermo Scientific) at 60° C. for 30 min. The reactions were cooled to 25° C. following incubation.

The absorbance for unstressed and stressed enzyme reactions and percent residual activities are shown in Table 8. VES33367M shows the highest percent residual activity together with VES33438M.

TABLE 8
Relative residual activity after 10 min at 94° C. at pH 4.5

| Samples | Unstressed | Stressed | % Residual Activity |
|---|---|---|---|
| VES33367M | 0.807 | 0.236 | 29 |
| VES33575 | 0.915 | 0.075 | 8 |
| VES33438M | 1.122 | 0.316 | 28 |
| VES35091M | 1.210 | 0.143 | 12 |
| SPEZYME ® HT ™ | 1.281 | 0.076 | 6 |
| SPEZYME ® SL ™ | 0.881 | 0.075 | 8 |

What is claimed is:

1. A non-naturally-occuring engineered α-amylase having at least 85% amino acid sequence identity relative to SEQ ID NO: 1, SEQ ID NO: 2, SEQ ID NO: 3 and/or SEQ ID NO: 4, and having α-amylase activity.

2. A nucleic acid encoding the non-naturally-occuring engineered α-amylase of claim 1.

3. An expression vector comprising the nucleic acid of claim 2.

4. A cell comprising the expression vector of claim 3.

5. A cell expressing the non-naturally-occuring engineered α-amylase of claim 1.

6. A formulated composition comprising the non-naturally-occuring engineered α-amylase of claim 1.

7. A method for saccharifying a composition comprising starch to produce a composition comprising glucose, wherein the method comprises:
(i) contacting the solution comprising starch with effective amount of the variant amylase of any of the claim 1; and
(ii) saccharifying the solution comprising starch to produce the composition comprising glucose; wherein the variant amylase catalyzes the saccharification of the starch solution to glucose.


SEQUENCE LISTING (18 sequences)

SEQ ID NO: 1 - protein, 483 aa, synthetic construct
AATNGTMMQYFEWYVPNDGQHWNKMKNDTAYLSSIGITALWIPPAYKGTSQADVGYGAYDLYDLGEFNQKGTVRTKYGTKAELKSAINTLHSKGIQVYGDVVMNHKAGADFTENVTAVEVNPSNRYQETSGEYNIQAWTGFNFPGRGTTYSNWKWQWFHFDGTDWDQSRSLSRIFKFHGKAWDWPVSSENGNYDYLMYADYDYDHPDVVNEMKKWGVWYANEVGLDGYRLDAVKHIKFSFLKDWVDNARAATGKEMFTVAEYWQNNLGEIENYLEKTGFNQSVFDVPLHYNFQAASSQGGAYDMRNILNGTVTSKQPTRSVTFVDNHDTQPGQALESTVQSWFKPLAYAFILTREAGYPAVFYGDMYGTKGTSGYEIPSLKTKIEPLLKARKDYAYGTQRDYIDNQDVIGWTREGDSTKAKSGLATVITDGPGGSKRMYVGKQNAGEVWYDITGNRTDTVTINADGYGEFHVNGGSVSVYVQK

SEQ ID NO: 2 - protein, 483 aa, synthetic construct
ASLNGTLMQYFEWYVPNDGQHWNRLQNDASYLSSVGITSLWIPPAYKGTSQNDVGYGAYDLYDLGEFNQKGTVRTKYGTKAELKSAINTLHSKGIQVYGDVVMNHKAGADATETVTAVEVNPNNRYQEISGEYQIQAWTGFNFPGRGNTYSNWKWHWYHFDGVDWDQSRSLSRIYKFDGKAWDWPVSNEYGNYDYLMYADYDYDHPDVVNEMKKWGTWYANEVNLDGFRIDAAKHIKFSFLGDWVQSVRTSTGKEMFTVAEYWQNNLGSLENYLEKSGNNHSVFDVPLHYNFYAASTQSGAYDMRNVLNGTVTAKYPTKSVTFVDNHDTQPGQSLESTVQTWFKPLAYAFILTREAGYPAVFYGDMYGTNGSTTYEIPALKSKIEPLLKARKDYAYGTQRDYIDNPDVIGWTREGDPSVAASGLATVITDGPGGSKRMYVGRQHAGETWHDITGNRSDPVTIHSDGYGEFHVNGGSVSIYVQK

SEQ ID NO: 3 - protein, 483 aa, synthetic construct
ASTNGTMMQYFEWYVPNDGQHWNRLQNDASYLSSVGITALWIPPAYKGTSQADVGYGAYDLYDLGEFNQKGTVRTKYGTKGELKSAINTLHSKGIQVYGDVVMNHKAGADATEDVTAVEVNPNNRYQEISGEYQIEAWTGFDFPGRGNTYSSFKWNWYHFDGVDWDQSRSLSRIYKFDGKAWDWPVSTEYGNYDYLMYADYDYDHPDVVNEMKKWGTWYANEVQLDGFRLDAVKHIKFSFLKDWVDNARAATGKEMFTVAEYWKNDLGALENYLEKTGFNQSVFDVPLHYNFHAASTQSGAYDMRNVLNGTVTAKYPTKSVTFVENHDTQPGQSLESTVQSWFKPLAYAFILTRESGYPAVFYGDMYGTKGTTTYEIPALKSKIEPLLKARKDYAYGTQRDYIDNQDVIGWTREGNTSKAKSGLATLITDGPGGSKRMYVGTQNAGEVWYDITGNRTDTVTINADGYGEFAVNGGSVSVWVQK

SEQ ID NO: 4 - protein, 482 aa, synthetic construct
ADNGTMMQYFEWYVPNDGQHWNKMKNDTAYLSSIGITAVWIPPAYKGTSQADVGYGAYDLYDLGEFNQKGTVRTKYGTKAELKSAITTLHSKGIQVYGDVVMNHKAGADFTENVTAVEVNPNNRYQEISGDYQIQAWTGFNFPGRGNTYSSFKWNWFHFDGTDYDQSRNLNRIYKFTGKAWDWPVSTEYGNYDYLMYADYDYDHPDVVNEMKKWGTWYANEVKLDGFRIDAAKHIKHSFLGDWVQSVRTSTGKEMFTVAEYWQNNLGSLENYLEKSGNNHSVFDVPLHYNFQAASSQGGAYDMRNILNGTVTSSQPTRSVTFVDNHDTQPGQALESTVQSWFKPLAYAFILTRESGYPAVFYGDMYGTKGTTGYEIPALKTKIEPLLKARKDFAYGTQRDYIDNPDVIGWTREGNTSKANSGLATLITDGPGGAKRMYVGTQNAGEVWYDLTGNRTDKVTIGSDGWATFNVNGGSVSVYVQQ

SEQ ID NO: 5 - DNA, 1452 bp, synthetic construct
  source 1..1452: mol_type = other DNA
gcagcgacgaatggaacgatgatgcaatattttgaatggtatgttccaaatgatggccagcattggaacaaaatgaagaatgatacggcttatttatcaagtatagggatcactgccctttggattcctccggcttataaagggacaagccaggcggatgttggctacggtgcatacgacctttatgacctgggagaatttaatcaaaaagggacggttcgaacgaaatatggaacaaaagctgaacttaaatctgccatcaatactcttcacagcaaaggcattcaagtatatggcgatgtcgtaatgaatcataaagccggagcggattttactgaaaatgtaacagctgtggaggtcaatccgtcaaaccgataccaggaaacatccggtgaatacaacatccaagcctggacgggctttaactttccaggtagaggcacaacctactccaactggaaatggcagtggtttcatttcgacggaacagattgggatcaatccagatcactatcaagaatctttaaattccatggaaaagcatgggattggccagtatcatcagaaaacggaaactatgattacttaatgtatgcggattacgattacgatcatccggatgttgtaaacgaaatgaaaaagtggggagtgtggtatgccaatgaagttggcctggatggatataggctggatgctgtgaaacatattaagttctccttccttaaagactgggtagataacgcgcgcgcggcgactggaaaagaaatgtttacagtggcagagtattggcaaaacaatcttggagaaattgaaaattacttagaaaaaacaggctttaatcagtcagtatttgatgtaccgctccactataactttcaggcagcctcttcacaaggcggtgcctatgatatgagaaatattttaaatggaacggttacttccaaacagccaacaagatcggtaacgtttgtagataatcatgatacacagccaggacaggctctggaatcaactgtgcaaagctggtttaaacctcttgcttatgctttcatattgacacgggaggcggggtatccagccgtgttttacggggatatgtacggaacaaaagggacaagcggctatgaaattcctagcttaaaaacaaagattgaacctttattaaaagcgagaaaagactacgcatacggtacccagcgggattatatcgacaatcaggatgtcataggctggacaagagaaggagattccacaaaagccaaatcaggactggcgactgtgattacggacggtccgggaggctcaaagcggatgtatgtcggtaaacaaaatgcaggagaagtgtggtatgatattacggggaatagaacggacacagtaactataaacgcggatggctatggcgaatttcatgtaaatggcggatctgtatccgtttatgtccagaaataa

SEQ ID NO: 6 - DNA, 1452 bp, synthetic construct
  source 1..1452: mol_type = other DNA
gcatcactgaatggaacgctgatgcaatattttgaatggtatgttccaaatgatggccagcattggaacagactgcagaatgatgcgtcatatttatcaagtgtggggatcacttcactttggattcctccggcttataaagggacaagccagaacgatgttggctacggtgcatacgacctttatgacctgggagaatttaatcaaaaagggacggttcgaacgaaatatggaacaaaagctgaacttaaatctgccatcaatactcttcacagcaaaggcattcaagtatatggcgatgtcgtaatgaatcataaagccggagcggatgcgactgaaacagtaacagctgtggaggtcaatccgaacaaccgataccaggaaatttccggtgaataccaaatccaagcctggacgggctttaactttccaggtagaggcaatacctactccaactggaaatggcattggtatcatttcgacggagtggattgggatcaatccagatcactatcaagaatctataaattcgatggaaaagcatgggattggccagtatcaaacgaatacggaaactatgattacttaatgtatgcggattacgattacgatcatccggatgttgtaaacgaaatgaaaaagtggggaacctggtatgccaatgaagttaacctggatggattcaggattgatgctgcgaaacatattaagttctccttccttggagactgggtacagtcagtccgcacctcgactggaaaagaaatgtttacagtggcagagtattggcaaaacaatcttggatcccttgaaaattacttagaaaaatccggcaataatcactcagtatttgatgtaccgctccactataacttttatgcagcctctacacaatcaggtgcctatgatatgagaaatgtgttaaatggaacggttactgcgaaatatccaacaaaatcggtaacgtttgtagataatcatgatacacagccaggacagtcactggaatcaactgtgcaaacatggtttaaacctcttgcttatgctttcatattgacacgggaggcggggtatccagccgtgttttacggggatatgtacggaacaaacgggtcaacaacatatgaaattcctgcgttaaaatcaaagattgaacctttattaaaagcgagaaaagactacgcatacggtacccagcgggattatatcgacaatccggatgtcatcggctggacaagagaaggagatccgtccgtggccgcgtcaggactggcgactgtgattacggacggtccgggaggctcaaagcggatgtatgtcggtagacaacatgcaggagaaacatggcatgatattacggggaatagatcagacccggtaactatacattcagatggctatggcgaatttcatgtaaatggcggatctgtatccatttatgtccagaaataa

SEQ ID NO: 7 - DNA, 1452 bp, synthetic construct
  source 1..1452: mol_type = other DNA
gcatcaacgaatggaacgatgatgcaatattttgaatggtatgttccaaatgatggccagcattggaacagactgcagaatgatgcgtcatatttatcaagtgtggggatcactgccctttggattcctccggcttataaagggacaagccaggcggatgttggctacggtgcgtacgacctttatgacctgggagaatttaatcaaaaagggacggttcgaacgaaatatggaacaaaaggcgaacttaaatctgccatcaatactcttcacagcaaaggcattcaagtatatggcgatgtcgtaatgaatcataaagccggagcggatgcgactgaagatgtaacagctgtggaggtcaatccgaacaaccgataccaggaaatttccggtgaataccaaatcgaagcctggacgggctttgactttccaggtagaggcaatacctactccagctttaaatggaactggtatcatttcgacggagtggattgggatcaatccagatcactatcaagaatctataaattcgatggaaaagcatgggattggccagtatcaaccgaatacggaaactatgattacttaatgtatgcggattacgattacgatcatccggatgttgtaaacgaaatgaaaaagtggggaacctggtatgccaatgaagttcagctggatggattcaggctggatgctgtgaaacatattaagttctccttccttaaagactgggtagataacgcgcgcgcggcgactggaaaagaaatgtttacagtggcagagtattggaaaaacgatcttggagcgcttgaaaattacttagaaaaaacaggctttaatcagtcagtatttgatgtaccgctccactataactttcatgcagcctctacacaatcaggtgcctatgatatgagaaatgtgttaaatggaacggttactgcgaaatatccaacaaaatcggtaacgtttgtagaaaatcatgatacacagccaggacagtcactggaatcaactgtgcaaagctggtttaaacctcttgcttatgctttcatattgacacgggagtctgggtatccagccgtgttttacggggatatgtacggaacaaaagggacaacaacatatgaaattcctgcgttaaaatcaaagattgaacctttattaaaagcgagaaaagactacgcatacggtacccagcgggattatatcgacaatcaggatgtcatcggctggacaagagaaggaaatacatccaaagccaaatcaggactggcgactcttattacggacggtccgggaggctcaaagcggatgtatgtcggtacacaaaatgcaggagaagtgtggtatgatattacggggaatagaacggacacagtaactataaacgcggatggctatggcgaatttgcggtaaatggcggatctgtatccgtttgggtccagaaataa

SEQ ID NO: 8 - DNA, 1449 bp, synthetic construct
  source 1..1449: mol_type = other DNA
gcagataatggaacgatgatgcaatattttgaatggtatgttccaaatgatggccagcattggaacaaaatgaagaatgatacggcttatttatcaagtatagggatcactgccgtttggattcctccggcttataaagggacaagccaggcggatgttggctacggtgcatacgacctttatgacctgggagaatttaatcaaaaagggacggttcgaacgaaatatggaacaaaagctgaacttaaatctgccattaccacacttcacagcaaaggcattcaagtatatggcgatgtcgtaatgaatcataaagcaggagcggattttactgaaaatgtaacagctgtggaggtcaatccgaacaaccgataccaggaaatttccggtgattaccaaatccaagcctggacgggctttaactttccaggtagaggcaatacctactccagctttaaatggaactggtttcatttcgacggaacagattatgatcaatccagaaatctaaacagaatctataaattcaccggaaaagcatgggattggccagtatcaaccgaatacggaaactatgattacttaatgtatgcggattacgattacgatcatccggatgttgtaaacgaaatgaaaaagtggggaacctggtatgccaatgaagttaagctggatggattcaggattgatgctgcgaaacatattaagcattccttccttggagactgggtacagtcagtccgcacctcgactggaaaagaaatgtttacagtggcagagtattggcaaaacaatcttggatcccttgaaaattacttagaaaaatccggcaataatcactcagtatttgatgtaccgctccactataactttcaggcagcctcttcacaaggcggtgcctatgatatgagaaatattttaaatggaacggttacttcctcacagccaacaagatcggtaacgtttgtagataatcatgatacacagccaggacaggctctggaatcaactgtgcaaagctggtttaaacctcttgcttatgctttcatattgacacgggagtctgggtatccagccgtgttttacggggatatgtacggaacaaaagggacaacaggctatgaaattcctgcgttaaaaacaaagattgaacctttattaaaagcgagaaaagactttgcatacggtacccagcgggattatatcgacaatccggatgttatcggctggacaagagaaggaaatacttccaaagccaattcaggactggcgactcttattacggacggtccgggaggcgctaagcggatgtatgtcggtacacaaaatgcaggagaagtttggtatgatctaacggggaatagaacggacaaagtaactataggttcagatggctgggcgacatttaatgtaaatggcggatctgtatccgtttatgtccagcagtag

SEQ ID NO: 9 - DNA, 1052 bp, synthetic construct
  source 1..1052: mol_type = other DNA
atcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctcgag

SEQ ID NO: 10 - DNA, 95 bp, synthetic construct
  source 1..95: mol_type = other DNA
gtcgctgataaacagctgacatcaatatcctattttttcaaaaaatattttaaaaagttgttgacttaaaagaagctaaatgttatagtaataaa

SEQ ID NO: 11 - DNA, 58 bp, Bacillus subtilis
  source 1..58: mol_type = other DNA
acagaatagtcttttaagtaagtctactctgaatttttttaaaaggagagggtaaaga

SEQ ID NO: 12 - DNA, 87 bp, Bacillus licheniformis
  source 1..87: mol_type = other DNA
atgaaacaacaaaaacggctttacgcccgattgctgacgctgttatttgcgctcatcttcttgctgcctcattctgcagcttcagca

SEQ ID NO: 13 - DNA, 48 bp, Bacillus licheniformis
  source 1..48: mol_type = other DNA
aagagcagagaggacggatttcctgaaggaaatccgtttttttatttt

SEQ ID NO: 14 - DNA, 2067 bp, Bacillus licheniformis
  source 1..2067: mol_type = other DNA
taacatctctcactgctgtgtgattttactcacggcatttggaacgccggctctcaacaaactttctgtagtgaaaatcatgaaccaaacggatcgtcggcctgattaacagctgaaagctgccgatcacaaacatccatagtcccgccggcttcagttcctcggagaaaaagcagaagctcccgacaaggaataaaaggccgatgagaaaatcgtttaatgtatgtagaactttgtatctttttttgaaaaagagttcatatcgattgttattgttttgcggcattgcttgatcactccaatccttttatttaccctgccggaagccggagtgaaacgccggtatacataggatttatgaattaggaaaacatatggggaaataaaccatccaggagtgaaaaatatgcggttattcatatgtgcatcgtgcctgttcggcttgattgttccgtcatttgaaacgaaagcgctgacgtttgaagaattgccggttaaacaagcttcaaaacaatgggaagttcaaatcggtaaagccgaagccggaaacggaatggcgaaaccggaaaaaggagcgtttcatacttatgctgtcgaaatcaaaaacattggacacgatgtggcttcggcggaaatttttgtctatcggaacgagcctaattcttcaacgaaattttcgctttggaacattcctcacgaaaatccggtttctttagccaaaagcttaaatcacggaagctctgtcaagcaccgcaatctgcttatggcagagaatgcgaccgaattggaagtggacatgatttggacggaaaaaggaagcgaaggcagacttttaaaggaaacgttcattttcaagggagatgaatcatgaagaaaaaatggccgttcatcgtcaacggtctttttttaatgacttaggcagccgatcgttcggccatacgatatcgaagcgacctcgaaccagcagagctcgtcacaaaacatttgcatttaaagaaaaatacaggatgttttcaccaatatttttctcaatgatgatacactattgacaagctgctactttgggagggtgtttccatagatgccgatgaagcaaaaacaccaaatgtgtcatgagagctctctctaatcgatataaaagtagggtgaaccggggttgtcaatctgtaaaagatctttttttatcccgtgatacgcttttggaattctgaatcttcaagaaagtccccagccttttgctgatcaatcgagaacaaaggatgatacatatgaaaagaatagataaaatctaccatcagctgctggataattttcgcgaaaagaatatcaatcagcttttaaagatacaagggaattcggctaaagaaatcgccgggcagctgcaaatggagcgttccaatgtcagctttgaattaaacaatctggttcgggccaaaaaggtgatcaagattaaaacgttccccgtccgctacatcccggtggaaattgttgaaaacgtcttgaacatcaaatggaattcagagttgatggaggttgaagaactgaggcggctggctgacggccaaaaaaagccggcgcgcaatatatccgccgatcccctcgagctcatgatcggggctaaagggagcttgaaaaaggcaatttctcaggcgaaagcggcagtcttttatcctccgcacggcttgcatatgctgctgctcgggccgacgggttcggggaaatcgctgtttgcgaatcggatctaccagttcgccgtttattctgacatattgaagcccgattccccgttcatcacattcaactgtgcagattactataacaaccctcaattattgctctctcaattgttcggacataaaaaagggtcttttacaggtgcgggtgaagacaaagcaggattagtcgagcaggcggacgggggcattctgtttatggatgaaatccatcgcctcccgccggaggggcaggaaatgctgttttatttcatagacagcggcacatacaacaggcttggtgaaacagagcataaacgaacggcaaaagtcctgtttatctgtgcgacaacag

SEQ ID NO: 15 - DNA, 2038 bp, Bacillus licheniformis
  source 1..2038: mol_type = other DNA
cgattaaacacggctaccgcagtattgataccgcagccatctacggtaatgaagagggggttgggcaaggaatccgcgaggggttgaaagaagccggcatttcaagagaagacctgtttgttacatcaaaggtctggaatgacgatttaggctatgacgaaacgattgcagcctatgaggcgagtctcgaaaagctcggacttgactaccttgatttatacctgatccactggcctgttgaaggacgctacaaagcggcgtggaaagcgcttgaaacactttatgaacaaggacgcgtaaaagcaatcggagtgagcaattttcagattcaccatctggaagacttgctgaaagatgccgccgtcaaaccggcgatcaaccaggttgagtatcatccgcggctgacgcagaaagagctgcaagcgttttgccgtgcgcacggcatccagctgcaagcatggtcgccgctgatgcaaggccaattgctcagccatccactgctgaaagatatcgcggacaagtacggcaagacaccggcccaagtcattttgcgctgggatttgcaaaacggggtcgttacgattccgaagtcgactaaagcggagcggattgcccaaaacgcggacatatttgattttgaactgaccaccgaggaaatgaagcaaattgacgcgctgaatgaaaacacccgtgtcggccctgatcccgataactttgacttttaacaaaacggccccgttcgacattcgaacggggctttaattgaattgtgcggttacaccgccggactccatcatcatcagttcttttttcatatccaatccgccccggtatcccgtgagctgcccgcttttaccgataacccgatggcaaggcaccaccattaacagcggatttgcgccgatcgccgcgcctactgcccgcacagcggcctgcttttcaatatgctcggcgatatcggaataggagcaagtgctgccgtaagggatttcggagagcgccttccacactgccagctgaaaaggcgtgccggcaaggtcgacaggaaagctgaaatgagttcgcttgccgttcaaatacgcctgcagctgctcggcgtattctgccaatcctttgtcatcccgaatgaaaactggctgtgtaaatcttttttcagcccaagcggccaaatcctcgaagccttgattccatccccctgtaaaacagagcccgcgggcagtcgccccaatgtgaatctgccaacctcggcaaataagcgtacgccagtatacgatttgatcgtccatatgtttacctccgtttcatttgccggtacgacgtcggcgattgcccagtcttctttttaaacaaagaggcaaaatattccgcattcgcaatgcctaccattgaagcgatttctgcgatcgatcgttctgaatgagcaagcaaatcgaccgctttctcaatccttttctgcaggatgtattctgccggcgagacgcctttgattcgtttaaatgtccgctgcaggtgaaaagggctgatatggcacctgtcagccaaagcttgcagagacagcggatcgcgataagattcctcgatgatttccaccacacgctgtgccagctcttcatccggcagcagcgccccggccggattgcagcgtttgcaggggcggtacccttctgataaagcatcttttgcattgaaaaagatctgcacattgtcgatttgcggaactctcgatttgcaggaagggcggcaaaatatgccggtcgttttgaccgcgtaataaaaaactccgtcataggcggaatcgttttccgtaatcgcccgccacatttcaggcgtcaatcgtgatttgctgttcatatcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaa

SEQ ID NO: 16 - protein, 483 aa, Bacillus licheniformis
ANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNSTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 17 - protein, 483 aa, Geobacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPR

SEQ ID NO: 18 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ